(19) United States
(12) Patent Application Publication
(10) Pub. No.: US 2004/0138238 A1
(43) Pub. Date: Jul. 15, 2004

(54) Substituted aminopyrimidine compounds as neurokinin antagonists

(76) Inventors: Dale S. Dhanoa, Wakefield, MA (US); Oren Becker, Mevaseret Zion (IL); Silvia Noiman, Herzliya (IL); Venkitasamy Kesavan, Woburn, MA (US); Anurag Sharadendu, Salem, NH (US); Pradyumna Mohanty, Woburn, MA (US); Dongli Chen, Chestnut Hill, MA (US); Srinivasa Rao Cheruku, Woburn, MA (US); Alexander Heifetz, Bnei-Brak (IL); Ori Kalid, Pardess Hanna (IL); Yael Marantz, Kadima (IL); Raphael Nudelman, Rehovot (IL); Sharon Sbacham, Alfey Menashe (IL); Shay Bar-Haim, Netanya (IL)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/626,085

(22) Filed: Jul. 24, 2003

Related U.S. Application Data
(60) Provisional application No. 60/401,952, filed on Aug. 8, 2002.
(60) Provisional application No. 60/414,998, filed on Oct. 1, 2002.
(60) Provisional application No. 60/465,379, filed on Apr. 25, 2003.

(51) Int. Cl. A61K 31/519; C07D 489/02

(52) U.S. Cl. 514/260.1; 514/265.1; 544/279; 544/281

(57) ABSTRACT

The invention discloses tachykinin receptor antagonists. The tachykinin family of receptors comprising the neurokinins substance P (SP), neurokinin A, and neurokinin B and related neuropeptides that are widely distributed in the peripheral and central nervous system. The invention discloses novel aminopyrimidine derivatives, synthesis and uses thereof for the treatment of diseases mediated directly or indirectly by the tachykinin receptors. These diseases include central nervous system disorders such as anxiety, pain, depression, emesis, in particular cancer chemotherapy induced emesis, respiratory and inflammatory bowel disease and other gastric disorders, asthma, schizophrenia, ophthalmic diseases such as glaucoma, ocular hypotension, neural injury, stroke, cardiac disorders, psoriasis, and migraine. Methods of preparation and novel intermediates and pharmaceutical salts thereof are also included.

### RELATED APPLICATIONS

This application claims the benefit of priority under 35 U.S.C. 119(e) to copending U.S. Provisional Application Nos. 60/401,952, filed on Aug. 8, 2002 as Docket No. 24591-501 PRO; 60/414,998, filed on Oct. 1, 2002 as Docket No. 24591-501 PRO B; and 60/465,379, filed on Apr. 25, 2003 as Docket No. 24591-501 PRO C, the entire contents of which are incorporated herein by reference.

### FIELD OF THE INVENTION

The invention generally relates to the field of neurokinin antagonists, and more particularly to new substituted aminopyrimidine compounds which are neurokinin antagonists and use of these compounds and their use in treatment and prevention of neurokinin conditions.

### BACKGROUND OF THE INVENTION

Major advances have been made in understanding the role of the mammalian tachykinin neuropeptides in the recent past. It is now well established that substance-P, neurokinin A (NKA), and neurokinin B (NKB), all of which share a common C-terminal sequence Phe-X-Gly-Leu-Met-NH₂, are widely distributed throughout the periphery and central nervous system (CNS) where they appear to interact with at least three receptor types referred to as NK₁, NK₂, and NK₃. Substance-P displays highest affinity for NK₁ receptors, whereas NKA and NKB bind preferentially to NK₂ and NK₃ receptors, respectively. All three receptors NK₁, NK₂, and NK₃ have been cloned and sequenced and shown to be members of the “super family” of G-protein coupled receptors (GPCRs.)

Considerable pre-clinical findings suggest the use of neurokinin receptor antagonists for the treatment of a wide range of biological diseases including migraine (Goadsby, P. J.; Hoskin, K. L.; Knight, Y. E. Neuroscience 86, 1, 337, 1998), arthritis (Von Sprecher, A.; Gerspacher, M.; Anderson, G. P., Drugs, 1, (1) 73, 1998), pain (Hill, R. G., In: The Tachykinin Receptors, ed. S. H. Buck, Humana Press Inc. Totowa, N.J., 471 (1994). Evidence also supports the involvement of tachykinin neuropeptides in a variety of biological activities including vasodilation, smooth muscle contraction, bronchoconstriction, immune system activation (inflammatory pain), and neurogenic inflammation. However, to date, a detailed understanding of the physiological role of these compounds has been severely hampered by a lack of selective, high affinity, metabolically stable neurokinin receptor antagonists that possess both good bioavailability and CNS penetration. Although several tachykinin receptor antagonists have been described, most have been developed through modifying and/or deleting one or more of the amino acids that comprise the endogenous mammalian tachykinins such that the resulting molecules are still peptides that possess poor pharmacokinetic properties and limited in vivo activities.

A number of high-affinity non-peptide antagonists have been reported, e.g., FK 888, CP 96345 and RP 67580 (NK₁ receptor antagonists), and SR 48969 (NK₂). Most of the non-peptide tachykinin receptor antagonists described to date directly or indirectly arose out of large compound collection screening using a robust radioligand binding assay as the primary screen. International Publication Numbers WO 93/01169, WO 93/01165, and WO 93/001160 discuss certain non-peptide tachykinin receptor antagonists.

Substance-P is widely distributed throughout the peripheral and central nervous systems. It is believed to mediate a variety of biological actions via an interaction with NK₁, NK₂, and NK₃ receptors, including smooth muscle contraction, pain transmission, neuronal excitation, saliva secretion, angiogenesis, bronchoconstriction, immune system activation, and neurogenic inflammation.

Accordingly, neurokinin receptor antagonists, e.g., compounds capable of antagonizing substance-P effects at NK₁ receptors will be useful in treating or preventing a variety of brain disorders such as pain, anxiety, panic, depression, schizophrenia, neuralgia, and addiction disorders; inflammatory diseases like arthritis, asthma, and psoriasis; gastrointestinal disorders including colitis, Crohn's disease, irritable bowel syndrome, and satiety; allergic responses such as eczema and rhinitis; vascular disorders such as angina and migraine; neuropathological disorders including Parkinson's disease, multiple sclerosis, and Alzheimer's disease; and ophthalmic diseases including scleroderma. Additionally, such compounds may be used as anti-angiogenic agents for treating conditions associated with aberrant neovascularization such as rheumatoid arthritis, atherosclerosis, and tumor cell growth; and as agents for imaging NK₁ receptors in vivo in conditions such as ulcerative colitis and Crohn's disease.

### SUMMARY OF THE INVENTION

The present invention relates to the discovery of new neurokinin antagonists that can be used for treating, preventing or curing neurokinin-related conditions. In particular, it has been found that certain substituted aminopyrimidine compounds are effective neurokinin antagonists. In an embodiment such neurokinin antagonist compounds include those having the formula

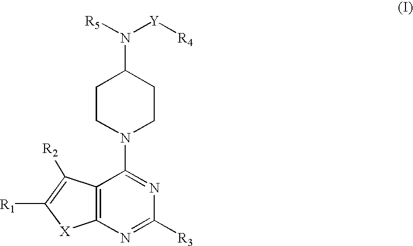

wherein

X may be S, O, C, NH, NR, or NCOR;

R₁ and R₂ each independently may be H; (C₁-C₇)alkyl; (C₁-C₇)cycloalkyl; (CH₂)ₙ—(C₁-C₇)cycloalkyl, aryl, substituted aryl, heteroaryl, or substituted heteroaryl; or R₁ and R₂, when joined by a single or multiple bonds, can form an aliphatic or an aromatic ring;

R₃ may be H, (C₁-C₄)alkyl, (C₁-C₆)cycloalkyl, aryl, substituted aryl, heteroaryl or substituted heteroaryl;

R₄ may be H, (C₁-C₅)alkyl, (C₁-C₆)cycloalkyl, aryl, substituted aryl, heteroaryl, or substituted heteroaryl; (CH₂)ₙ₋aryl or (CH₂)ₙ-heteroaryl, where n is 1, 2 or 3;

Y may be CH₂, hydroxycyclohexyl,

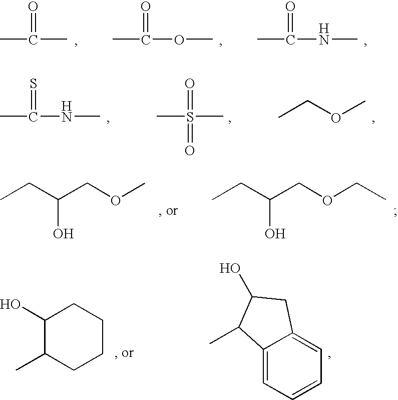

with the proviso that when R₅ forms a heterocyclic ring with the nitrogen to which it is attached, Y is attached to the heterocylic ring;

R₅ may be H; (C₁-C₅)alkyl; (C₁-C₆)cycloalkyl, aryl, substituted aryl, heteroaryl or substituted heteroaryl; (CH₂)ₙ₋aryl or (CH₂)ₙ-heteroaryl, where n is 1, 2 or 3;

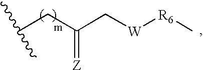

where m is 1, 2, 3, 4 or 5; or R₅, taken with the nitrogen to which it is attached, forms a five or six membered heterocyclic ring to which Y is attached, of the structure

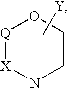

where X is a methylene (—CH₂—) or carbonyl group

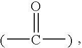

and Q is a methylene group or not present;

Z may be H, H; O, H and OH, O-alkyl where alkyl is (C₁-C₆)alkyl, (C₁-C₆)cycloalkyl, O-alkylaryl, O-benzyl, O—CO-aryl, N—Me, N-acyl, N-aryl, N-aroyl, N—SO₂-alkyl, or N—SO₂-aryl;

W may be C, O, NH, NR; and

R₆ may be H; (C₁-C₅)alkyl; (C₁-C₆)cycloalkyl, aryl, substituted aryl, heteroaryl, or substituted heteroaryl; (CH₂)ₙ₋aryl or (CH₂)ₙ-heteroaryl; where n is 1, 2 or 3; and pharmaceutically acceptable salts and/or esters thereof.

The aryl group may be desirably phenyl, naphthyl, or biphenyl.

Suitable heteroaryl groups include thiazole, oxazole, benzothiazole, benzoxazole, pyrazole, indole, and indazole.

Substituted aryl groups include mono-, di-, or tri-substituted phenyl, naphthyl, or biphenyl with methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy. More desirably, substituted aryl groups include mono-, di-, or tri-substituted thiazole, oxazole, benzothiazole, benzoxazole, pyrazole, indole, and indazole.

The substituents may be, e.g., methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy. In particular embodiments, X is sulfur.

In an embodiment, neurokinin antagonists of the invention include those where R₂ is aryl and R₁ is either H or methyl. R₅ may be H, Y may be CH₂, and R₄ may be H; R₅ may be H, and Y may be an ester linkage, and R₄ may be alkyl; and R₅ may be H and Y and R₄ may join to form a conjugated ring system.

In another embodiment neurokinin antagonist compounds of the invention include those having formula II:

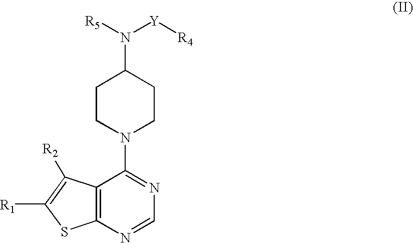

wherein

R₁ may be H or CH₃;

R₂ may be CH₃ or substituted or unsubstituted aryl;

R₃ may be H; (C₁-C₅)alkyl; or substituted or unsubstituted aryl;

Y may be CH₂, hydroxycyclohexyl,

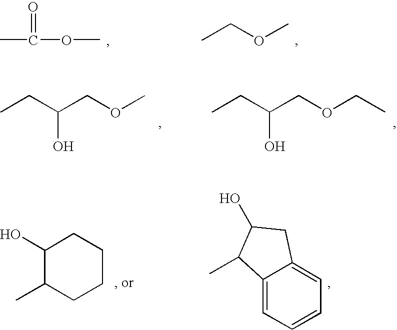

with the proviso that when R₅ forms a heterocyclic ring with the nitrogen to which it is attached, Y is attached to the heterocylic ring;

R₄ may be substituted or unsubstituted aryl, e.g., mono-, di- or trisubstituted with halo, trihalomethyl, hydroxyl, alkoxy (e.g., methoxy), or with a dioxole ring; and pharmaceutically acceptable salts and/or esters thereof; and

R₅ may be H; (C₁-C₅)alkyl; (C₁-C₆)cycloalkyl, aryl, substituted aryl, heteroaryl or substituted heteroaryl; (CH₂)ₙ₋aryl or (CH₂)ₙ-heteroaryl, where n is 1, 2 or 3; or R₅, taken with the nitrogen to which it is attached, forms a five or six membered heterocyclic ring to which Y is attached, of the structure

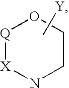

where X is a methylene (—CH₂—) or carbonyl group

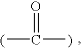

and Q is a methylene group or not present.

In another embodiment, neurokinin antagonist compounds of the invention include those having formula III:

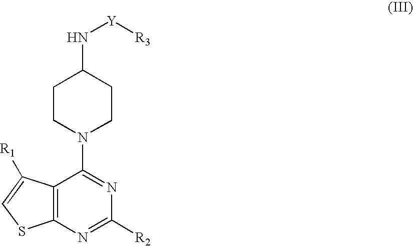

wherein

R₁ may be selected from the group consisting of substituted or unsubstituted aryl and substituted or unsubstituted heteroaryl;

R₂ may be H, (C₁-C₅)alkyl, (C₁-C₆)cycloalkyl, aryl, substituted aryl, heteroaryl, or substituted heteroaryl; (CH₂)ₙ₋aryl or (CH₂)ₙ-heteroaryl, where n is 1, 2 or 3; and pharmaceutically acceptable salts and/or esters thereof.

R₃ may be selected from the group consisting of substituted or unsubstituted aryl and substituted or unsubstituted heteroaryl; and

Y maybe

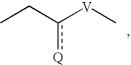

wherein Q or V is O, OH, S, or SH.

The aryl group may be desirably phenyl, naphthyl, or biphenyl.

Suitable heteroaryl groups include thiazole, oxazole, benzothiazole, benzoxazole, pyrazole, indole, and indazole.

Substituted aryl groups include mono-, di-, or tri-substituted phenyl, naphthyl, or biphenyl with methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy. More desirably, substituted aryl groups include mono-, di-, or tri-substituted thiazole, oxazole, benzothiazole, benzoxazole, pyrazole, indole, and indazole.

The substituents may be, e.g., methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy.

Another aspect of the invention is a pharmaceutical composition comprising an amount of a compound of the invention effective to treat respiratory disorders in a mammal suffering therefrom, and a pharmaceutically acceptable carrier.

Another aspect of the invention is a method for treating respiratory disorders in a mammal such as a human comprising administering a therapeutically effective amount of a compound of the invention.

Another aspect of the invention is a pharmaceutical composition comprising an amount of a compound of the invention effective to treat inflammation in a mammal suffering therefrom, and a pharmaceutically acceptable carrier.

Another aspect of the invention is a method for treating inflammation in a mammal such as a human comprising administering a therapeutically effective amount of a compound of the invention.

Another aspect of the invention is a pharmaceutical composition comprising an amount of a compound of the invention effective to treat gastrointestinal disorders in a mammal suffering therefrom, and a pharmaceutically acceptable carrier.

Another aspect of the invention is a method for treating gastrointestinal disorders in a mammal such as a human comprising administering a therapeutically effective amount of a compound of the invention.

Another aspect of the invention is a pharmaceutical composition comprising an amount of a compound of the invention effective to treat eye diseases such as dry eye and conjunctivitis in a mammal suffering therefrom, and a pharmaceutically acceptable carrier.

Another aspect of the invention is a method for treating eye diseases in a mammal such as a human comprising administering a therapeutically effective amount of a compound of the invention.

Another aspect of the invention is a pharmaceutical composition comprising an amount of a compound of the invention effective to treat allergies in a mammal suffering therefrom, and a pharmaceutically acceptable carrier.

Another aspect of the invention is a method for treating allergies in a mammal such as a human comprising administering a therapeutically effective amount of a compound of the invention.

Another aspect of the invention is a pharmaceutical composition comprising an amount of a compound of the invention effective to treat diseases of the central nervous system in a mammal suffering therefrom, and a pharmaceutically acceptable carrier.

Another aspect of the invention is a method for treating diseases of the central nervous system in a mammal such as a human comprising administering a therapeutically effective amount of a compound of the invention.

Another aspect of the invention is a pharmaceutical composition comprising an amount of a compound of the invention effective to treat migraine in a mammal suffering therefrom, and a pharmaceutically acceptable carrier.

Another aspect of the invention is a method for treating migraine in a mammal such as a human comprising administering a therapeutically effective amount of a compound of the invention.

Another aspect of the invention is a pharmaceutical composition comprising an amount of compound of the invention effective to treat pain arising from neurogenic inflammation or inflammatory pain.

Another aspect of the invention is a method for treating pain such as pain arising from neurogenic inflammation in inflammatory pain status.

Another aspect of the invention is a pharmaceutical composition comprising an amount of a compound of the invention effective in treating conditions associated with aberrant neovascularization: rheumatoid arthritis, atherosclerosis, and tumor cell growth.

Another aspect of the invention is a method of treating conditions associated with aberrant neovascularization: rheumatoid arthritis, atherosclerosis, and tumor cell growth.

Another aspect of the invention is using the compounds as imaging agents for imaging NK₁ receptors in vivo.

In particular embodiments, compounds of the invention include 2-[1-(5-Phenyl-thieno[2,3-d]pyrimidin-4-yl)-piperidin-4-ylamino]-cyclohexanol; 2-[1-(6-Methyl-5-phenyl-thieno[2,3-d]pyrimidin-4-yl)-piperidin-4-ylamino]-cyclohexanol; 1-[1-(6-Methyl-5-phenyl-thieno[2,3-d]pyrimidin-4-yl)-piperidin-4-ylamino]-indan-2-ol; 5-Methoxy-2-{[1-(5-phenyl-thieno[2,3-d]pyrimidin-4-yl)-piperidin-4-ylamino]-methyl}-phenol; Bis-(2-fluoro-benzyl)-[1-(5-phenyl-thieno[2,3-d]pyrimidin-4-yl)-piperidin-4-yl]-amine; 1-{1-[5-(4-Bromo-phenyl)-thieno[2,3-d]pyrimidin-4-yl]-piperidin-4-ylamino}-indan-2-ol; 1-[1-(5-p-Tolyl-thieno[2,3-d]pyrimidin-4-yl)-piperidin-4-ylamino]-indan-2-ol; 2-Fluoro-6-{[1-(6-methyl-5-phenyl-thieno[2,3-d]pyrimidin-4-yl)-piperidin-4-ylamino]-methyl}-phenol; 2-({1-[5-(4-Bromo-phenyl)-thieno[2,3-d]pyrimidin-4-yl]-piperidin-4-ylamino}-methyl)-6-fluoro-phenol; 2-Fluoro-6-{[1-(5-p-tolyl-thieno[2,3-d]pyrimidin-4-yl)-piperidin-4-ylamino]-methyl}-phenol; 1-[1-(5-Phenyl-thieno[2,3-d]pyrimidin-4-yl)-piperidin-4-ylamino]-indan-2-ol; 1-(4-Fluoro-phenoxy)-3-[1-(5-phenyl-thieno[2,3-d]pyrimidin-4-yl)-piperidin-4-ylamino]-propan-2-ol; 1-[1-(5-Phenyl-thieno[2,3-d]pyrimidin-4-yl)-piperidin-4-ylamino]-3-(4-trifluoromethoxy-phenoxy)-propan-2-ol; 1-(3,4-Difluoro-phenoxy)-3-{1-[5-(4-fluoro-phenyl)-thieno[2,3-d]pyrimidin-4-yl]-piperidin-4-ylamino}-propan-2-ol; 1-(4-Methoxy-phenoxy)-3-[1-(5-phenyl-thieno[2,3-d]pyrimidin-4-yl)-piperidin-4-ylamino]-propan-2-ol; 2-{1-[5-(4-Bromo-phenyl)-thieno[2,3-d]pyrimidin-4-yl]-piperidin-4-ylamino}-cyclohexanol; 2-[1-(5-p-Tolyl-thieno[2,3-d]pyrimidin-4-yl)-piperidin-4-ylamino]-cyclohexanol; 2-[1-(6-Methyl-5-phenyl-thieno[2,3-d]pyrimidin-4-yl)-piperidin-4-ylamino]-cyclohexanol; 1-(4-Chloro-phenoxy)-3-[1-(5-phenyl-thieno[2,3-d]pyrimidin-4-yl)-piperidin-4-ylamino]-propan-2-ol; 1-Phenoxy-3-[1-(5-phenyl-thieno[2,3-d]pyrimidin-4-yl)-piperidin-4-ylamino]-propan-2-ol; 1-Benzyloxy-3-[1-(5-phenyl-thieno[2,3-d]pyrimidin-4-yl)-piperidin-4-ylamino]-propan-2-ol; 1-(Benzo[1,3]dioxol-5-yloxy)-3-[1-(5-phenyl-thieno[2,3-d]pyrimidin-4-yl)-piperidin-4-ylamino]-propan-2-ol; 1-(Benzo[1,3]dioxol-5-ylmethoxy)-3-[1-(5-phenyl-thieno[2,3-d]pyrimidin-4-yl)-piperidin-4-ylamino]-propan-2-ol; 1-(3,4-Difluoro-phenoxy)-3-[1-(5-phenyl-thieno[2,3-d]pyrimidin-4-yl)-piperidin-4-ylamino]-propan-2-ol; 1-(2-Chloro-4-methoxy-phenoxy)-3-[4-(5-phenyl-thieno[2,3-d]pyrimidin-4-yl)-cyclohexylamino]-propan-2-ol; 1-(3,4-Dimethoxy-phenoxy)-3-[4-(5-phenyl-thieno[2,3-d]pyrimidin-4-yl)-cyclohexylamino]-propan-2-ol; 1-(3,4-Dichloro-phenoxy)-3-[1-(5-phenyl-thieno[2,3-d]pyrimidin-4-yl)-piperidin-4-ylamino]-propan-2-ol; 1-(3-Chloro-4-fluoro-phenoxy)-3-[1-(5-phenyl-thieno[2,3-d]pyrimidin-4-yl)-piperidin-4-ylamino]-propan-2-ol; 1-(2,4-Difluoro-phenoxy)-3-[1-(5-phenyl-thieno[2,3-d]pyrimidin-4-yl)-piperidin-4-ylamino]-propan-2-ol; 1-(3,5-Difluoro-phenoxy)-3-[1-(5-phenyl-thieno[2,3-d]pyrimidin-4-yl)-piperidin-4-ylamino]-propan-2-ol; 1-(3,5-Bis-trifluoromethyl-phenoxy)-3-[1-(5-phenyl-thieno[2,3-d]pyrimidin-4-yl)-piperidin-4-ylamino]-propan-2-ol; 1-(Benzo[1,3]dioxol-5-yloxy)-3-[1-(5-methyl-thieno[2,3-d]pyrimidin-4-yl)-piperidin-4-ylamino]-propan-2-ol; 1-(Benzo[1,3]dioxol-5-yloxy)-3-(1-thieno[2,3-d]pyrimidin-4-yl-piperidin-4-ylamino)-propan-2-ol; [2-Hydroxy-3-(4-methoxy-phenoxy)-propyl]-[1-(5-phenyl-thieno[2,3-d]pyrimidin-4-yl)-piperidin-4-yl]-ammonium; chloride; [3-(2-Chloro-4-methoxy-phenoxy)-2-hydroxy-propyl]-[4-(5-phenyl-thieno[2,3-d]pyrimidin-4-yl)-cyclohexyl]-ammonium; chloride; [3-(3,4-Dimethoxy-phenoxy)-2-hydroxy-propyl]-[4-(5-phenyl-thieno[2,3-d]pyrimidin-4-yl)-cyclohexyl]-amonium; chloride; [3-(3,4-Dichloro-phenoxy)-2-hydroxy-propyl]-[1-(5-phenyl-thieno[2,3-d]pyrimidin-4-yl)-piperidin-4-yl]-ammonium; chloride; [3-(2,4-Difluoro-phenoxy)-2-hydroxy-propyl]-[1-(5-phenyl-thieno[2,3-d]pyrimidin-4-yl)-piperidin-4-yl]-ammonium; chloride; 1-[1-(5-Phenyl-thieno[2,3-d]pyrimidin-4-yl)-piperidin-4-ylamino]-3-p-tolyloxy-propan-2-ol; [2-Hydroxy-3-(4-trifluoromethyl-phenoxy)-propyl]-[1-(5-phenyl-thieno[2,3-d]pyrimidin-4-yl)-piperidin-4-yl]-ammonium; chloride; [3-(4-Chloro-phenoxy)-2-hydroxy-propyl]-[1-(5-phenyl-thieno[2,3-d]pyrimidin-4-yl)-piperidin-4-yl]-ammonium; chloride; 1-(3,4-Dimethoxy-phenoxy)-3-[1-(5-phenyl-thieno[2,3-d]pyrimidin-4-yl)-piperidin-4-ylamino]-propan-2-ol; 1-(4-Chloro-3-methoxy-phenoxy)-3-[1-(5-phenyl-thieno[2,3-d]pyrimidin-4-yl)-piperidin-4-ylamino]-propan-2-ol; 4-{4-[2-(4-Fluoro-phenoxymethyl)-morpholin-4-yl]-piperidin-1-yl}-5-phenyl-thieno[2,3-d]pyrimidine; 4-{4-[2-(Benzo[1,3]dioxol-5-yloxymethyl)-morpholin-4-yl]-piperidin-1-yl}-5-phenyl-thieno[2,3-d]pyrimidine; 6-(Benzo[1,3]dioxol-5-yloxymethyl)-4-[1-(5-phenyl-thieno[2,3-d]pyrimidin-4-yl)-piperidin-4-yl]-morpholin-3-one; 1-(2-Chloro-4-methoxy-phenoxy)-3-[1-(5-phenyl-thieno[2,3-d]pyrimidin-4-yl)-piperidin-4-ylamino]-propan-2-ol; and 1-(3,4-Dimethoxy-phenoxy)-3-[1-(5-phenyl-thieno[2,3-d]pyrimidin-4-yl)-piperidin-4-ylamino]-propan-2-ol.

Processes for preparing the compounds and novel intermediates are also included in the invention, as discussed further below.

### DETAILED DESCRIPTION OF THE INVENTION

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

Definitions

For convenience, certain terms used in the specification, examples, and appended claims are collected here.

“G-protein coupled receptor” (GPCR) includes the NK₁, NK₂, and NK₃ receptors.

“Neurokinin” includes substance-P, neurokinin A, and neurokinin B.

“Neurokinin antagonist” includes compounds having such effect at the NK₁, NK₂, and NK₃ receptors.

“Treating”, includes any effect, e.g., lessening, reducing, modulating, or eliminating, that results in the improvement of the condition, disease, disorder, etc.

“Alkyl” includes saturated aliphatic groups, including straight-chain alkyl groups (e.g., methyl, ethyl, propyl, butyl, pentyl, hexyl, heptyl, octyl, nonyl, decyl), branched-chain alkyl groups (e.g., isopropyl, tert-butyl, isobutyl), cycloalkyl (e.g., alicyclic) groups (e.g., cyclopropyl, cyclopentyl, cyclohexyl, cycloheptyl, cyclooctyl), alkyl substituted cycloalkyl groups, and cycloalkyl substituted alkyl groups. “Alkyl” further includes alkyl groups which have oxygen, nitrogen, sulfur or phosphorous atoms replacing one or more hydrocarbon backbone carbon atoms. In certain embodiments, a straight chain or branched chain alkyl has six or fewer carbon atoms in its backbone (e.g., C₁-C₆ for straight chain, C₃-C₆ for branched chain), and more preferably four or fewer. Likewise, preferred cycloalkyls have from three to eight carbon atoms in their ring structure, and more preferably have five or six carbons in the ring structure. “C₁-C₆” includes alkyl groups containing one to six carbon atoms.

The term “alkyl” also includes both “unsubstituted alkyls” and “substituted alkyls”, the latter of which refers to alkyl moieties having substituents replacing a hydrogen on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g. phenylmethyl (benzyl)). “Alkyl” also includes the side chains of natural and unnatural amino acids.

“Aryl” includes groups with aromaticity, including 5- and 6-membered “unconjugated”, or single-ring, aromatic groups that may include from zero to four heteroatoms, as well as “conjugated”, or multicyclic, systems with at least one aromatic ring. Examples of aryl groups include benzene, phenyl, pyrrole, furan, thiophene, thiazole, isothiazole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a multicyclic system (e.g., tetralin, methylenedioxyphenyl).

“Alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl), branched-chain alkenyl groups, cycloalkenyl (e.g., alicyclic) groups (e.g., cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “alkenyl” further includes alkenyl groups which include oxygen, nitrogen, sulfur or phosphorous atoms replacing one or more hydrocarbon backbone carbons. In certain embodiments, a straight chain or branched chain alkenyl group has six or fewer carbon atoms in its backbone (e.g., C₂-C₆ for straight chain, C₃-C₆ for branched chain.) Likewise, cycloalkenyl groups may have from three to eight carbon atoms in their ring structure, and more preferably have five or six carbons in the ring structure. The term “C₂-C₆” includes alkenyl groups containing two to six carbon atoms.

The term “alkenyl” also includes both “unsubstituted alkenyls” and “substituted alkenyls”, the latter of which refers to alkenyl moieties having substituents replacing a hydrogen on one or more hydrocarbon backbone carbon atoms. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight-chain alkynyl groups (e.g., ethynyl, propynyl, butynyl, pentynyl, hexynyl, heptynyl, octynyl, nonynyl, decynyl), branched-chain alkynyl groups, and cycloalkyl or cycloalkenyl substituted alkynyl groups. The term “alkynyl” further includes alkynyl groups having oxygen, nitrogen, sulfur or phosphorous atoms replacing one or more hydrocarbon backbone carbons. In certain embodiments, a straight chain or branched chain alkynyl group has six or fewer carbon atoms in its backbone (e.g., C₂-C₆ for straight chain, C₃-C₆ for branched chain). The term “C₂-C₆” includes alkynyl groups containing two to six carbon atoms.

The term “alkynyl” also includes both “unsubstituted alkynyls” and “substituted alkynyls”, the latter of which refers to alkynyl moieties having substituents replacing a hydrogen on one or more hydrocarbon backbone carbon atoms. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” includes an alkyl group, as defined above, but having from one to ten, more preferably from one to six, carbon atoms in its backbone structure. “Lower alkenyl” and “lower alkynyl” have chain lengths of, for example, 2-5 carbon atoms.

“Acyl” includes compounds and moieties which contain the acyl radical (CH₃CO—) or a carbonyl group. “Substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Acylamino” includes moieties wherein an acyl moiety is bonded to an amino group. For example, the term includes alkylcarbonylamino, arylcarbonylamino, carbamoyl and ureido groups.

“Aroyl” includes compounds and moieties with an aryl or heteroaromatic moiety bound to a carbonyl group. Examples of aroyl groups include phenylcarboxy, naphthyl carboxy, etc.

“Alkoxyalkyl”, “alkylaminoalkyl” and “thioalkoxyalkyl” include alkyl groups, as described above, which further include oxygen, nitrogen or sulfur atoms replacing one or more hydrocarbon backbone carbon atoms, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, and trichloromethoxy.

The terms “heterocyclyl” or “heterocyclic group” include closed ring structures, e.g., 3- to 10-, or 4- to 7-membered rings, which include one or more heteroatoms. Heterocyclyl groups can be saturated or unsaturated and include pyrrolidine, oxolane, thiolane, piperidine, piperazine, morpholine, lactones, lactams such as azetidinones and pyrrolidinones, sultams, sultones, and the like. The heterocyclic ring can be substituted at one or more positions with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety.

The term “thiocarbonyl” or “thiocarboxy” includes compounds and moieties which contain a carbon connected with a double bond to a sulfur atom.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or heteroatoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

“Polycyclyl” or “polycyclic radical” refers to two or more cyclic rings (e.g., cycloalkyls, cycloalkenyls, cycloalkynyls, aryls and/or heterocyclyls) in which two or more carbons are common to two adjoining rings. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminocarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

“Heteroatom” includes atoms of any element other than carbon or hydrogen. Examples of heteroatoms include nitrogen, oxygen, sulfur and phosphorus.

It will be noted that the structure of some of the compounds of the invention includes asymmetric carbon atoms. It is to be understood accordingly that the isomers arising from such asymmetry (e.g., all enantiomers and diastereomers) are included within the scope of the invention, unless indicated otherwise. Such isomers can be obtained in substantially pure form by classical separation techniques and by stereochemically controlled synthesis. Furthermore, the structures and other compounds and moieties discussed in this application also include all tautomers thereof. Alkenes can include either the E- or Z-geometry, where appropriate.

Combination therapy” (or “co-therapy”) includes the administration of a compound of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

An “anionic group,” as used herein, refers to a group that is negatively charged at physiological pH. Preferred anionic groups include carboxylate, sulfate, sulfonate, sulfinate, sulfamate, tetrazolyl, phosphate, phosphonate, phosphinate, or phosphorothioate or functional equivalents thereof. “Functional equivalents” of anionic groups are intended to include bioisosteres, e.g., bioisosteres of a carboxylate group. Bioisosteres encompass both classical bioisosteric equivalents and non-classical bioisosteric equivalents. Classical and non-classical bioisosteres are known in the art (see, e.g., Silverman, R. B. The Organic Chemistry of Drug Design and Drug Action, Academic Press, Inc.: San Diego, Calif., 1992, pp.19-23). A particularly preferred anionic group is a carboxylate.

The term “heterocyclic group” is intended to include closed ring structures in which one or more of the atoms in the ring is an element other than carbon, for example, nitrogen, or oxygen or sulfur. Heterocyclic groups can be saturated or unsaturated and heterocyclic groups such as pyrrole and furan can have aromatic character. They include fused ring structures such as quinoline and isoquinoline. Other examples of heterocyclic groups include pyridine and purine. Heterocyclic groups can also be substituted at one or more constituent atoms with, for example, a halogen, a lower alkyl, a lower alkenyl, a lower alkoxy, a lower alkylthio, a lower alkylamino, a lower alkylcarboxyl, a nitro, a hydroxyl, —CF₃, —CN, or the like.

Substituted aminopyrimidine compounds of the invention are effective neurokinin antagonists. In one embodiment such neurokinin antagonist compounds include those having the formula

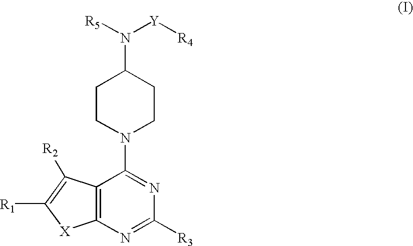

wherein

X may be S, O, C, NH, NR, or NCOR;

R₁ and R₂ each independently may be H; (C₁-C₇)alkyl; (C₁-C₇)cycloalkyl; (CH₂)ₙ—(C₁-C₇)cycloalkyl, aryl, substituted aryl, heteroaryl, or substituted heteroaryl; or R₁ and R₂, when joined by a single or multiple bonds, can form an aliphatic or an aromatic ring;

R₃ may be H, (C₁-C₄)alkyl, (C₁-C₆)cycloalkyl, aryl, substituted aryl, heteroaryl or substituted heteroaryl;

R₄ may be H, (C₁-C₅)alkyl, (C₁-C₆)cycloalkyl, aryl, substituted aryl, heteroaryl, or substituted heteroaryl; (CH₂)ₙ₋aryl or (CH₂)ₙ-heteroaryl, where n is 1, 2 or 3;

Y may be CH₂, hydroxycyclohexyl,

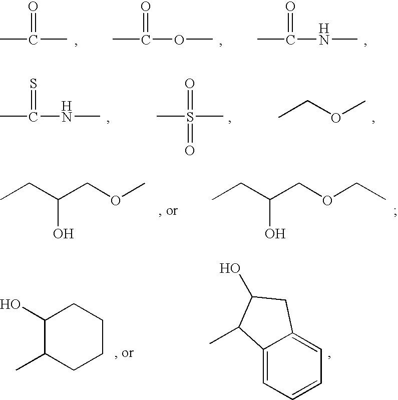

with the proviso that when R₅ forms a heterocyclic ring with the nitrogen to which it is attached, Y is attached to the heterocylic ring;

R₅ may be H; (C₁-C₅)alkyl; (C₁-C₆)cycloalkyl, aryl, substituted aryl, heteroaryl or substituted heteroaryl; (CH₂)ₙ₋aryl or (CH₂)ₙ-heteroaryl, where n is 1, 2 or 3;

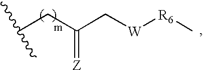

where m is 1, 2, 3, 4 or 5; or R₅, taken with the nitrogen to which it is attached, forms a five or six membered heterocyclic ring to which Y is attached, of the structure

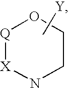

where X is a methylene (—CH₂—) or carbonyl group

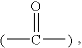

and Q is a methylene group or not present;

Z may be H, H; O, H and OH, O-alkyl where alkyl is (C₁-C₆)alkyl, (C₁-C₆)cycloalkyl, O-alkylaryl, O-benzyl, O—CO-aryl, N—Me, N-acyl, N-aryl, N-aroyl, N—SO₂-alkyl, or N—SO₂-aryl;

W may be C, O, NH, NR; and

R₆ may be H; (C₁-C₅)alkyl; (C₁-C₆)cycloalkyl, aryl, substituted aryl, heteroaryl, or substituted heteroaryl; (CH₂)ₙ₋aryl or (CH₂)ₙ-heteroaryl; where n is 1, 2 or 3; and pharmaceutically acceptable salts and/or esters thereof.

The aryl group may be desirably phenyl, naphthyl, or biphenyl.

Suitable heteroaryl groups include thiazole, oxazole, benzothiazole, benzoxazole, pyrazole, indole, and indazole.

Substituted aryl groups include mono-, di-, or tri-substituted phenyl, naphthyl, or biphenyl with methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy. More desirably, substituted aryl groups include mono-, di-, or tri-substituted thiazole, oxazole, benzothiazole, benzoxazole, pyrazole, indole, and indazole.

The substituents may be, e.g., methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy. In particular embodiments, X is sulfur.

In an embodiment, neurokinin antagonists of the invention include those where R₂ is aryl and R₁ is either H or methyl. R₅ may be H, Y may be CH₂, and R₄ may be H; R₅ may be H, and Y may be an ester linkage, and R₄ may be alkyl; and R₅ may be H and Y and R₄ may join to form a conjugated ring system.

In another embodiment neurokinin antagonist compounds of the invention include those having formula II:

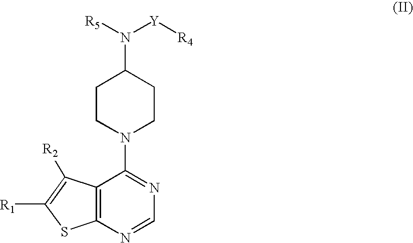

wherein

R₁ may be H or CH₃;

R₂ may be CH₃ or substituted or unsubstituted aryl;

R₃ may be H; (C₁-C₅)alkyl; or substituted or unsubstituted aryl;

Y may be CH₂, hydroxycyclohexyl,

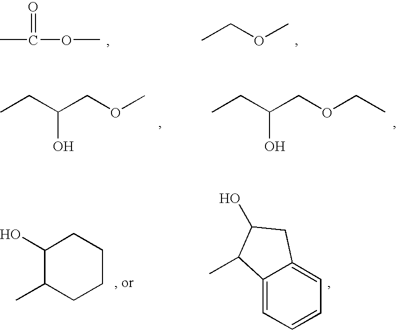

with the proviso that when R₅ forms a heterocyclic ring with the nitrogen to which it is attached, Y is attached to the heterocylic ring;

R₄ may be substituted or unsubstituted aryl, e.g., mono-, di- or trisubstituted with halo, trihalomethyl, hydroxyl, alkoxy (e.g., methoxy), or with a dioxole ring; and pharmaceutically acceptable salts and/or esters thereof; and

R₅ may be H; (C₁-C₅)alkyl; (C₁-C₆)cycloalkyl, aryl, substituted aryl, heteroaryl or substituted heteroaryl; (CH₂)ₙ₋aryl or (CH₂)ₙ-heteroaryl, where n is 1, 2 or 3; or R₅, taken with the nitrogen to which it is attached, forms a five or six membered heterocyclic ring to which Y is attached, of the structure

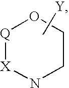

where X is a methylene (—CH₂—) or carbonyl group

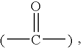

and Q is a methylene group or not present.

In another embodiment, neurokinin antagonist compounds of the invention include those having formula III:

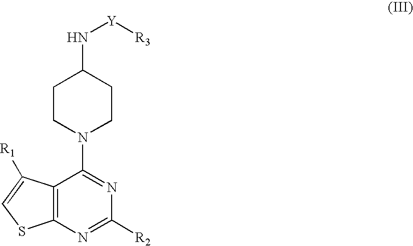

wherein

R₁ may be selected from the group consisting of substituted or unsubstituted aryl and substituted or unsubstituted heteroaryl;

R₂ may be H, (C₁-C₅)alkyl, (C₁-C₆)cycloalkyl, aryl, substituted aryl, heteroaryl, or substituted heteroaryl; (CH₂)ₙ₋aryl or (CH₂)ₙ-heteroaryl, where n is 1, 2 or 3; and pharmaceutically acceptable salts and/or esters thereof.

R₃ may be selected from the group consisting of substituted or unsubstituted aryl and substituted or unsubstituted heteroaryl; and

Y may be

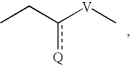

wherein Q or V is O, OH, S, or SH.

The aryl group may be desirably phenyl, naphthyl, or biphenyl.

Suitable heteroaryl groups include thiazole, oxazole, benzothiazole, benzoxazole, pyrazole, indole, and indazole.

Substituted aryl groups include mono-, di-, or tri-substituted phenyl, naphthyl, or biphenyl with methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy. More desirably, substituted aryl groups include mono-, di-, or tri-substituted thiazole, oxazole, benzothiazole, benzoxazole, pyrazole, indole, and indazole.

The substituents may be, e.g., methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy.

In particular embodiments, compounds of the invention include 2-[1-(5-Phenyl-thieno[2,3-d]pyrimidin-4-yl)-piperidin-4-ylamino]-cyclohexanol; 2-[1-(6-Methyl-5-phenyl-thieno[2,3-d]pyrimidin-4-yl)-piperidin-4-ylamino]-cyclohexanol; 1-[1-(6-Methyl-5-phenyl-thieno[2,3-d]pyrimidin-4-yl)-piperidin-4-ylamino]-indan-2-ol; 5-Methoxy-2-{[1-(5-phenyl-thieno[2,3-d]pyrimidin-4-yl)-piperidin-4-ylamino]-methyl}-phenol; Bis-(2-fluoro-benzyl)-[1-(5-phenyl-thieno[2,3-d]pyrimidin-4-yl)-piperidin-4-yl]-amine; 1-{1-[5-(4-Bromo-phenyl)-thieno[2,3-d]pyrimidin-4-yl]-piperidin-4-ylamino}-indan-2-ol; 1-[1-(5-p-Tolyl-thieno[2,3-d]pyrimidin-4-yl)-piperidin-4-ylamino]-indan-2-ol; 2-Fluoro-6-{[1-(6-methyl-5-phenyl-thieno[2,3-d]pyrimidin-4-yl)-piperidin-4-ylamino]-methyl}-phenol; 2-({1-[5-(4-Bromo-phenyl)-thieno[2,3-d]pyrimidin-4-yl]-piperidin-4-ylamino}-methyl)-6-fluoro-phenol; 2-Fluoro-6-{[1-(5-p-tolyl-thieno[2,3-d]pyrimidin-4-yl)-piperidin-4-ylamino]-methyl}-phenol; 1-[1-(5-Phenyl-thieno[2,3-d]pyrimidin-4-yl)-piperidin-4-ylamino]-indan-2-ol; 1-(4-Fluoro-phenoxy)-3-[1-(5-phenyl-thieno[2,3-d]pyrimidin-4-yl)-piperidin-4-ylamino]-propan-2-ol; 1-[1-(5-Phenyl-thieno[2,3-d]pyrimidin-4-yl)-piperidin-4-ylamino]-3-(4-trifluoromethoxy-phenoxy)-propan-2-ol; 1-(3,4-Difluoro-phenoxy)-3-{1-[5-(4-fluoro-phenyl)-thieno[2,3-d]pyrimidin-4-yl]-piperidin-4-ylamino}-propan-2-ol; 1-(4-Methoxy-phenoxy)-3-[1-(5-phenyl-thieno[2,3-d]pyrimidin-4-yl)-piperidin-4-ylamino]-propan-2-ol; 2-{1-[5-(4-Bromo-phenyl)-thieno[2,3-d]pyrimidin-4-yl]-piperidin-4-ylamino}-cyclohexanol; 2-[1-(5-p-Tolyl-thieno[2,3-d]pyrimidin-4-yl)-piperidin-4-ylamino]-cyclohexanol; 2-[1-(6-Methyl-5-phenyl-thieno[2,3-d]pyrimidin-4-yl)-piperidin-4-ylamino]-cyclohexanol; 1-(4-Chloro-phenoxy)-3-[1-(5-phenyl-thieno[2,3-d]pyrimidin-4-yl)-piperidin-4-ylamino]-propan-2-ol; 1-Phenoxy-3-[1-(5-phenyl-thieno[2,3-d]pyrimidin-4-yl)-piperidin-4-ylamino]-propan-2-ol; 1-Benzyloxy-3-[1-(5-phenyl-thieno[2,3-d]pyrimidin-4-yl)-piperidin-4-ylamino]-propan-2-ol; 1-(Benzo[1,3]dioxol-5-yloxy)-3-[1-(5-phenyl-thieno[2,3-d]pyrimidin-4-yl)-piperidin-4-ylamino]-propan-2-ol; 1-(Benzo[1,3]dioxol-5-ylmethoxy)-3-[1-(5-phenyl-thieno[2,3-d]pyrimidin-4-yl)-piperidin-4-ylamino]-propan-2-ol; 1-(3,4-Difluoro-phenoxy)-3-[1-(5-phenyl-thieno[2,3-d]pyrimidin-4-yl)-piperidin-4-ylamino]-propan-2-ol; 1-(2-Chloro-4-methoxy-phenoxy)-3-[4-(5-phenyl-thieno[2,3-d]pyrimidin-4-yl)-cyclohexylamino]-propan-2-ol; 1-(3,4-Dimethoxy-phenoxy)-3-[4-(5-phenyl-thieno[2,3-d]pyrimidin-4-yl)-cyclohexylamino]-propan-2-ol; 1-(3,4-Dichloro-phenoxy)-3-[1-(5-phenyl-thieno[2,3-d]pyrimidin-4-yl)-piperidin-4-ylamino]-propan-2-ol; 1-(3-Chloro-4-fluoro-phenoxy)-3-[1-(5-phenyl-thieno[2,3-d]pyrimidin-4-yl)-piperidin-4-ylamino]-propan-2-ol; 1-(2,4-Difluoro-phenoxy)-3-[1-(5-phenyl-thieno[2,3-d]pyrimidin-4-yl)-piperidin-4-ylamino]-propan-2-ol; 1-(3,5-Difluoro-phenoxy)-3-[1-(5-phenyl-thieno[2,3-d]pyrimidin-4-yl)-piperidin-4-ylamino]-propan-2-ol; 1-(3,5-Bis-trifluoromethyl-phenoxy)-3-[1-(5-phenyl-thieno[2,3-d]pyrimidin-4-yl)-piperidin-4-ylamino]-propan-2-ol; 1-(Benzo[1,3]dioxol-5-yloxy)-3-[1-(5-methyl-thieno[2,3-d]pyrimidin-4-yl)-piperidin-4-ylamino]-propan-2-ol; 1-(Benzo[1,3]dioxol-5-yloxy)-3-(1-thieno[2,3-d]pyrimidin-4-yl-piperidin-4-ylamino)-propan-2-ol; [2-Hydroxy-3-(4-methoxy-phenoxy)-propyl]-[1-(5-phenyl-thieno[2,3-d]pyrimidin-4-yl)-piperidin-4-yl]-ammonium; chloride; [3-(2-Chloro-4-methoxy-phenoxy)-2-hydroxy-propyl]-[4-(5-phenyl-thieno[2,3-d]pyrimidin-4-yl)-cyclohexyl]-ammonium; chloride; [3-(3,4-Dimethoxy-phenoxy)-2-hydroxy-propyl]-[4-(5-phenyl-thieno[2,3-d]pyrimidin-4-yl)-cyclohexyl]-ammonium; chloride; [3-(3,4-Dichloro-phenoxy)-2-hydroxy-propyl]-[1-(5-phenyl-thieno[2,3-d]pyrimidin-4-yl)-piperidin-4-yl]-ammonium; chloride; [3-(2,4-Difluoro-phenoxy)-2-hydroxy-propyl]-[1-(5-phenyl-thieno[2,3-d]pyrimidin-4-yl)-piperidin-4-yl]-ammonium; chloride; 1-[1-(5-Phenyl-thieno[2,3-d]pyrimidin-4-yl)-piperidin-4-ylamino]-3-p-tolyloxy-propan-2-ol; [2-Hydroxy-3-(4-trifluoromethyl-phenoxy)-propyl]-[1-(5-phenyl-thieno[2,3-d]pyrimidin-4-yl)-piperidin-4-yl]-ammonium; chloride; [3-(4-Chloro-phenoxy)-2-hydroxy-propyl]-[1-(5-phenyl-thieno[2,3-d]pyrimidin-4-yl)-piperidin-4-yl]-ammonium; chloride; 1-(3,4-Dimethoxy-phenoxy)-3-[1-(5-phenyl-thieno[2,3-d]pyrimidin-4-yl)-piperidin-4-ylamino]-propan-2-ol; 1-(4-Chloro-3-methoxy-phenoxy)-3-[1-(5-phenyl-thieno[2,3-d]pyrimidin-4-yl)-piperidin-4-ylamino]-propan-2-ol; 4-{4-[2-(4-Fluoro-phenoxymethyl)-morpholin-4-yl]-piperidin-1-yl}-5-phenyl-thieno[2,3-d]pyrimidine; 4-{4-[2-(Benzo[1,3]dioxol-5-yloxymethyl)-morpholin-4-yl]-piperidin-1-yl}-5-phenyl-thieno[2,3-d]pyrimidine; 6-(Benzo[1,3]dioxol-5-yloxymethyl)-4-[1-(5-phenyl-thieno[2,3-d]pyrimidin-4-yl)-piperidin-4-yl]-morpholin-3-one; 1-(2-Chloro-4-methoxy-phenoxy)-3-[1-(5-phenyl-thieno[2,3-d]pyrimidin-4-yl)-piperidin-4-ylamino]-propan-2-ol; and 1-(3,4-Dimethoxy-phenoxy)-3-[1-(5-phenyl-thieno[2,3-d]pyrimidin-4-yl)-piperidin-4-ylamino]-propan-2-ol.

Another aspect of the invention is a pharmaceutical composition comprising an amount of a compound of the invention effective to treat respiratory disorders in a mammal suffering therefrom, and a pharmaceutically acceptable carrier.

Another aspect of the invention is a method for treating respiratory disorders in a mammal such as a human comprising administering a therapeutically effective amount of a compound of the invention.

Another aspect of the invention is a pharmaceutical composition comprising an amount of a compound of the invention effective to treat inflammation in a mammal suffering therefrom, and a pharmaceutically acceptable carrier.

Another aspect of the invention is a method for treating inflammation in a mammal such as a human comprising administering a therapeutically effective amount of a compound of the invention.

Another aspect of the invention is a pharmaceutical composition comprising an amount of a compound of the invention effective to treat gastrointestinal disorders in a mammal suffering therefrom, and a pharmaceutically acceptable carrier.

Another aspect of the invention is a method for treating gastrointestinal disorders in a mammal such as a human comprising administering a therapeutically effective amount of a compound of the invention.

Another aspect of the invention is a pharmaceutical composition comprising an amount of a compound of the invention effective to treat eye diseases such as dry eye and conjunctivitis in a mammal suffering therefrom, and a pharmaceutically acceptable carrier.

Another aspect of the invention is a method for treating eye diseases in a mammal such as a human comprising administering a therapeutically effective amount of a compound of the invention.

Another aspect of the invention is a pharmaceutical composition comprising an amount of a compound of the invention effective to treat allergies in a mammal suffering therefrom, and a pharmaceutically acceptable carrier.

Another aspect of the invention is a method for treating allergies in a mammal such as a human comprising administering a therapeutically effective amount of a compound of the invention.

Another aspect of the invention is a pharmaceutical composition comprising an amount of a compound of the invention effective to treat diseases of the central nervous system in a mammal suffering therefrom, and a pharmaceutically acceptable carrier.

Another aspect of the invention is a method for treating diseases of the central nervous system in a mammal such as a human comprising administering a therapeutically effective amount of a compound of the invention.

Another aspect of the invention is a pharmaceutical composition comprising an amount of a compound of the invention effective to treat migraine in a mammal suffering therefrom, and a pharmaceutically acceptable carrier.

Another aspect of the invention is a method for treating migraine in a mammal such as a human comprising administering a therapeutically effective amount of a compound of the invention.

Another aspect of the invention is a pharmaceutical composition comprising an amount of compound of the invention effective to treat pain arising from neurogenic inflammation or inflammatory pain.

Another aspect of the invention is a method for treating pain such as pain arising from neurogenic inflammation in inflammatory pain status.

Another aspect of the invention is a pharmaceutical composition comprising an amount of a compound of the invention effective in treating conditions associated with aberrant neovascularization: rheumatoid arthritis, atherosclerosis, and tumor cell growth.

Another aspect of the invention is a method of treating conditions associated with aberrant neovascularization: rheumatoid arthritis, atherosclerosis, and tumor cell growth.

Another aspect of the invention is using the compounds as imaging agents for imaging NK₁ receptors in vivo.

Processes for preparing the compounds and novel intermediates are also included in the invention.

The compounds of the invention are valuable for treating a wide variety of clinical conditions which are characterized by the presence of an excess of tachykinin, e.g., substance P, activity.

Thus, for example, an excess of neurokinin activity is implicated in a variety of disorders of the central nervous system. Such disorders include eating disorders, schizophrenia, neuralgia, and addiction disorders; obsessive compulsive disorders, panic disorders, sexual dysfunctions caused by the central nervous system and disturbances in sleep and the absorption of food, alcoholism, pain, memory deficits, unipolar depression, dysthymia, bipolar depression, treatment-resistant depression, depression in the medically ill, panic disorder, obsessive-compulsive disorder, eating disorders, social phobia, premenstrual dysphoric disorder, mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, e.g., specific animal phobias, social phobias, stress disorders including post-traumatic stress disorder and acute stress disorder, and generalized anxiety disorders; schizophrenia and other psychotic disorders, for example, schizophreniform disorders, schizoaffective disorders, delusional disorders, brief psychotic disorders, shared psychotic disorders and psychotic disorders with delusions or hallucinations; delirium, dementia, and amnestic and other cognitive or neurodegenerative disorders, such as Alzheimer's disease, senile dementia, dementia of the Alzheimer's type, vascular dementia, and other dementias, for example, due to HIV disease, head trauma, Parkinson's disease, Huntington's disease, Pick's disease, Creutzfeldt-Jakob disease, or due to multiple etiologies; Parkinson's disease and other extra-pyramidal movement disorders such as medication-induced movement disorders, for example, neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor; substance-related disorders arising from the use of alcohol, amphetamines (or amphetamine-like substances) caffeine, cannabis, cocaine, hallucinogens, inhalants and aerosol propellants, nicotine, opioids, phenylglycidine derivatives, sedatives, hypnotics, and anxiolytics, which substance-related disorders include dependence and abuse, intoxication, withdrawal, intoxication delirium, withdrawal delirium, persisting dementia, psychotic disorders, mood disorders, anxiety disorders, sexual dysfunction and sleep disorders; epilepsy; Down's syndrome; demyelinating diseases such as MS and ALS and other neuropathological disorders such as peripheral neuropathy, for example diabetic and chemotherapy-induced neuropathy, and postherpetic neuralgia, trigeminal neuralgia, segmental or intercostal neuralgia and other neuralgias; and cerebral vascular disorders due to acute or chronic cerebrovascular damage such as cerebral infarction, subarachnoid hemorrhage or cerebral edema.

Neurokinin activity is also involved in nociception and pain. The compounds of the invention will therefore be useful in preventing or treating diseases and conditions in which pain predominates, including soft tissue and peripheral damage, such as acute trauma, osteoarthritis, rheumatoid arthritis, musculo-skeletal pain, particularly after trauma, spinal pain, myofascial pain syndromes, headache, episiotomy pain, and burns; deep and visceral pain, such as heart pain, muscle pain, eye pain, orofacial pain, for example, odontalgia, abdominal pain, gynecological pain, for example, dysmenorrhea, and labor pain; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; pain associated with carcinoma, often referred to as cancer pain; central nervous system pain, such as pain due to spinal cord or brain stem damage; low back pain; sciatica; ankylosing spondylitis, gout; and scar pain.

Neurokinin antagonists may also be useful in treating respiratory diseases, particularly those associated with excess mucus secretion, such as chronic obstructive airways disease, bronchopneumonia, chronic bronchitis, cystic fibrosis and asthma, adult respiratory distress syndrome, and bronchospasm; inflammatory diseases such as inflammatory bowel disease, psoriasis, fibrositis, osteoarthritis, rheumatoid arthritis, pruritis and sunburn; allergies such as eczema and rhinitis; hypersensitivity disorders such as poison ivy; ophthalmic diseases such as conjunctivitis, vernal conjunctivitis, and the like; ophthalmic conditions associated with cell proliferation such as proliferative vitreoretinopathy; cutaneous diseases such as contact dermatitis, atopic dermatitis, urticaria, and other eczematoid dermatitis.

Neurokinin antagonists may also be useful in treating neoplasms, including breast tumors, neuroganglioblastomas and small cell carcinomas such as small cell lung cancer.

Neurokinin antagonists may also be useful in treating gastrointestinal (GI) disorders, including inflammatory disorders and diseases of the GI tract such as gastritis, gastroduodenal ulcers, gastric carcinomas, gastric lymphomas, disorders associated with the neuronal control of viscera, ulcerative colitis, Crohn's disease, irritable bowel syndrome and emesis, including acute, delayed or anticipatory emesis such as emesis induced by chemotherapy, radiation, toxins, viral or bacterial infections, pregnancy, vestibular disorders, for example, motion sickness, vertigo, dizziness and Meniere's disease, surgery, migraine, variations in intercranial pressure, gastro-esophageal reflux disease, acid indigestion, over indulgence in food or drink, acid stomach, waterbrash or regurgitation, heartburn, for example, episodic, nocturnal or meal-induced heartburn, and dyspepsia.

Neurokinin antagonists may also be useful in treating a variety of other conditions including stress related somatic disorders; reflex sympathetic dystrophy such as shoulder/hand syndrome; adverse immunological reactions such as rejection of transplanted tissues and disorders related to immune enhancement or suppression such as systemic lupus erythematosus; plasma extravasation resulting from cytokine chemotherapy, disorders of bladder function such as cystitis, bladder detrusor hyper-reflexia and incontinence; fibrosing and collagen diseases such as scleroderma and eosinophilic fascioliasis; disorders of blood flow caused by vasodilation and vasospastic diseases such as angina, vascular headache, migraine and Reynaud's disease; and pain or nociception attributable to or associated with any of the foregoing conditions, especially pain transmission in migraine.

The compounds of the invention are also valuable in treating a combination of the above conditions, in particular in the treatment of combined post-operative pain and post-operative nausea and vomiting.

The compounds of the invention are particularly useful in treating emesis, including acute, delayed or anticipatory emesis, such as emesis induced by chemotherapy, radiation, toxins, pregnancy, vestibular disorders, motion, surgery, migraine, and variations in intercranial pressure. Most especially, the compounds of the invention are useful in treating emesis induced by antineoplastic (cytotoxic) agents including those routinely used in cancer chemotherapy.

Examples of such chemotherapeutic agents include alkylating agents like nitrogen mustards, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; and cytotoxic antibiotics.

Particular examples of chemotherapeutic agents are described, for example, by D. J. Stewart in “Nausea and Vomiting: Recent Research and Clinical-Advances”, Eds. J. Kucharczyk, et al., CRC Press Inc., Boca Raton, Fla., USA (1991), pages 177-203, especially page 188. Commonly used chemotherapeutic agents include cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, and chlorambucil [R. J. Gralla, et al., Cancer Treatment Reports, 68(1), 163-172 (1984)].

The compounds of the invention are also useful in treating emesis induced by radiation including radiation therapy such as in cancer treatment, or radiation sickness; and in the treatment of post-operative nausea and vomiting.

For treating certain conditions it may be desirable to employ the compound of the invention in conjunction with another pharmacologically active agent. The compounds of the invention may be presented together with another therapeutic agent as a combined preparation for simultaneous, separate or sequential use for the relief of emesis. Such combined preparations may be, for example, in the form of a twin pack.

A further aspect of the invention comprises compounds of the invention in combination with a 5-HT₃ antagonist, such as ondansetron, granisetron, tropisetron or zatisetron, or other anti-emetic medicaments, for example, dexamethasone or a dopamine antagonist such as metoclopramide. Additionally, the compounds of the invention may be administered in combination with an anti-inflammatory corticosteroid, such as dexamethasone. Furthermore, the compounds of the invention may be administered in combination with a chemotherapeutic agent such as an alkylating agent, antimetabolite, mitotic inhibitor or cytotoxic antibiotic, as described above. In general, the currently available dosage forms of the known therapeutic agents for use in such combinations will be suitable.

The compounds of the invention are also particularly useful for treating pain or nociception and/or inflammation and disorders associated therewith, such as neuropathy, e.g., diabetic and chemotherapy-induced neuropathy, postherpetic and other neuralgias, asthma, osteoarthritis, rheumatoid arthritis and headache, including migraine, acute or chronic tension headache, cluster headache, temporomandibular pain, and maxillary sinus pain. The invention further provides the compounds of the invention for therapeutic use.

According to a further or alternative aspect, the invention provides compounds of the invention for use in the manufacture of a medicament for the treatment or prevention of physiological disorders associated with neurokinin excess.

The invention also provides methods for treating or preventing physiological disorders associated with neurokinin excess, which method comprises administration to a patient in need thereof of a tachykinin-reducing amount of a compound of the invention or a composition comprising a compound of the invention.

For treating certain conditions it may be desirable to employ a compound according to the invention in conjunction with another pharmacologically active agent. For example, for treating respiratory diseases such as asthma, the compound of the invention may be used in conjunction with a bronchodilator, such as a β₂-adrenergic receptor antagonist or tachykinin antagonist which acts at NK-2 receptors. The compound of the invention and the bronchodilator may be administered to a patient simultaneously, sequentially or in combination. For treating conditions that require antagonism of both neurokinin-1 and neurokinin-2, including disorders associated with bronchoconstriction and/or plasma extravasation in airways, such as asthma, chronic bronchitis, airways disease, or cystic fibrosis, the compound of the invention may be used in conjunction with a tachykinin antagonist which acts at neurokinin-2 receptors, or with tachykinin receptor antagonist which acts at both neurokinin-1 and neurokinin-2 receptors.

Likewise, the compounds of the invention may be employed with a leukotriene antagonist, such as a leukotriene D₄ antagonist such as disclosed in European patent specification nos. 0 480 717 and 0 604 114, and in U.S. Pat. Nos. 4,859,692 and 5,270,324. This combination is particularly useful in treating respiratory diseases such as asthma, chronic bronchitis and cough.

The invention accordingly provides a method for treating a respiratory disease, e.g., asthma, which method comprises administration to a patient in need thereof of an effective amount of the compound of the invention and an effective amount of a bronchodilator. The invention also provides a composition comprising the compound of the invention, a bronchodilator, and a pharmaceutically acceptable carrier.

For treating or preventing migraine, the compounds of the invention may be used in conjunction with other anti-migraine agents, such as ergotamines or 5-HT₁ agonists, especially sumatriptan or rizatriptan. Likewise, for treating behavioral hyperalgesia, the compounds of the invention may be used in conjunction with an antagonist of N-methyl D-aspartate (NMDA), such as dizocilpine. For treating or preventing inflammatory conditions in the lower urinary tract, especially cystitis, the compounds of the invention may be used in conjunction with an anti-inflammatory agent such as a bradykinin receptor antagonist. The invention also provides a composition comprising a compound of the invention, a bronchodilator, and a pharmaceutically acceptable carrier.

For treating or preventing pain or nociception, the compounds of the invention may be used in conjunction with other analgesics, such as acetaminophen (paracetamol), aspirin and other NSAIDs and, in particular, opioid analgesics, especially morphine. Specific anti-inflammatory agents include diclofenac, ibuprofen, indomethacin, ketoprofen, naproxen, piroxicam and sulindac. Suitable opioid analgesics of use in conjunction with a compound of the invention include morphine, codeine, dihydrocodeine, diacetylmorphine, hydrocodone, hydromorphone, levorphanol, oxymorphone, afenantil, buprenorphine, butorphanol, fentanyl, sufentanyl, meperidine, methadone, nalbuphine, propoxyphene and pentazocine; or a pharmaceutically acceptable salt thereof. Preferred salts of these opioid analgesics include morphine sulfate, morphine hydrochloride, morphine tartrate, codeine phosphate, codeine sulfate, dihydrocodeine bitartrate, diacetylmorphine hydrochloride, hydrocodone bitartrate, hydromorphone hydrochloride, levorphanol tartrate, oxymorphone hydrochloride, afenantil hydrochloride, buprenorphine hydrochloride, butorphanol tartrate, fentanyl citrate, meperidine hydrochloride, methadone hydrochloride, nalbuphine hydrochloride, propoxyphene hydrochloride, propoxyphene napsylate (2-naphthalenesulphonic acid (1:1) monohydrate), and pentazocine hydrochloride.

Therefore, in a further aspect of the invention, a pharmaceutical composition is provided comprising a compound of the invention and an analgesic, together with at least one pharmaceutically acceptable carrier or excipient.

In a further or alternative aspect of the invention, a product is provided comprising a compound of the invention and an analgesic as a combined preparation for simultaneous, separate or sequential use in the treatment or prevention of pain or nociception.

It will be further appreciated that for treating or preventing depression and/or anxiety, the compounds of the invention may be used in combination with an antidepressant agent or anti-anxiety agent. Suitable classes of antidepressant agents of use in the invention include: norepinephrine reuptake inhibitors, selective serotonin reuptake inhibitors, monoamine oxidase inhibitors, reversible monoamine oxidase inhibitors, serotonin and noradrenaline reuptake inhibitors, corticotropin releasing factor (CRF) antagonists, β-adrenoreceptor antagonists and atypical antidepressants. Another class of antidepressant agent of use in the invention is noradrenergic and specific serotonergic antidepressants, such as mirtazapine. Suitable examples of norepinephrine reuptake inhibitors include amitripdyline, clomipramine, doxepine, imipramine, trimipramine, amoxapine, desipramine, maprotiline, nortriptyline, reboxetine and protriptyline and pharmaceutically acceptable salts thereof. Suitable examples of selective serotonin reuptake inhibitors include fluoxetine, fluvoxamine, paroxetine, and sertraline and pharmaceutically acceptable salts thereof. Suitable examples of monoamine oxidase inhibitors include isocarboxazid, phenelzine, tranylcypromain and selegiline, and pharmaceutically acceptable salts thereof. Suitable examples of reversible monoamine oxidase inhibitors include moclobemide, and pharmaceutically acceptable salts thereof. Suitable examples of serotonin and noradrenaline reuptake inhibitors include venlafaxine, and pharmaceutically acceptable salts thereof. Suitable examples of corticotropin releasing factor (CRF) antagonists include those compounds described in International Patent Specification Nos. WO 94/13643, WO 94/13644, WO 94/13661, WO 94/13676 and WO 94/13677. Suitable examples of atypical antidepressants include bupropion, lithium, nefazoedone, sibutramine, trazodone and viloxazine, and pharmaceutically acceptable salts thereof. Other antidepressants of use in the invention include adinozolam, alaproclate, amineptine, amitryptyline/chlordiazepoxide combination, atipamezole, azamianserin, bazinaprine, fefuraline, bifemelane, binodaline, bipenamol, brofaromine, bupropion, caroxazone, cericlamine, cianopramine, cimoxatone, citalopram, clemeprol, clovoxamine, dasepinil, deanol, demexiptiline, dibenzepin, dothiepin, droxidopa, enefexine, setazolam, etoperidone, femoxetine, fengabine, fezolamine, fluotracen, idazoxan, indalpine, indeloxazine, iprindole, levoprotiline, litoxetine, lofepramine, medifoxamine, metapramine, metralindole, mianserin, milnacipran, minaprine, mirtazapine, montirelin, nebracetam, nefopam, nialamide, nomifensine, norfluoxetine, orotirelin, oxaflozane, pinazepam, pirindole, pizotyline, ritaserin, rolipram, sercloremine, setiptiline, sibutramine, sulbutiamine, sulpride, teniloxazine, thozalinone, thymoliberin, tianeptine, tiflucarbine, tofenacin, tofisopam, toloxatone, tomoxetine, veralipride, viqualine, zimelidine, and zometapine, and pharmaceutically acceptable salts thereof, and St. John's wort herb, or Hypericum perforatum, or extracts thereof. Preferred antidepressant agents include selective serotonin reuptake inhibitors, in particular, fluoxetine, fluvoxamine, paroxetine, and sertraline and pharmaceutically acceptable salts thereof.

Suitable classes of anti-anxiety agents of use in the invention include benzodiazepines and 5-HT_{1A} agonists or antagonists, especially 5-HT_{1A} partial agonists, and corticotropin releasing factor (CRF) antagonists. In addition to benzodiazepines, other suitable classes of anti-anxiety agents are nonbenzodiazepine sedative-hypnotic drugs such as zolpidem; mood-stabilizing drugs such as clobazam, gabapentin, lamotrigine, loreclezole, oxcarbamazepine, stiripentol and vigabatrin; and barbiturates. Suitable benzodiazepines of use in the invention include alprazolam, chlordizepoxide, clonazepam, chlorazepate, diazepam, halazepam, is lorezepam, oxazepam and prazepam, and pharmaceutically acceptable salts thereof. Suitable examples of 5-HT_{1A} agonists or antagonists of use in the invention include, in particular, the 5-HT_{1A} partial agonists buspirone, flesinoxan, gepirone, ipsapirone and pindolol, and pharmaceutically acceptable salts thereof. Suitable examples of corticotropin releasing factor (CRF) antagonists include those compounds described in International Patent Specification Nos. WO 94/13643, WO 94/13644, WO 94/13661, WO 94/13676 and WO 94/13677. Another class of anti-anxiety agent of use in the invention are compounds having muscarinic cholinergic activity. Suitable compounds in this class include m 1 muscarinic cholinergic receptor antagonists such as those compounds described in European Patent Specification Nos. 0 709 093, 0 709 094 and 0 773 021 and International Patent Specification No. WO 96/12711. Another class of anti-anxiety agent of use in the invention are compounds acting on ion channels. Suitable compounds in this class include carbamazepine, lamotrigine and valproate, and pharmaceutically acceptable salts thereof.

Therefore, in a further aspect of the invention, a pharmaceutical composition is provided comprising a compound of the invention and an antidepressant or an anti-anxiety agent, together with at least one pharmaceutically acceptable carrier or excipient.

Suitable antipsychotic agents of use in combination with the compounds of the invention include phenothiazines, e.g., chlorpromazine, mesoridazine, thioridazine, acetophenazine, fluphenazine, perphenazine and trifluoperazine; thioxanthenes, e.g., chlorprothixene or thiothixene; heterocyclic dibenzazepines, e.g., clozapine or olanzapine; butyrophenones, e.g., haloperidol; diphenylbutylpiperidines, e.g., pimozide; and indolones, e.g., molindolene. Other antipsychotic agents include loxapine, sulpiride and risperidone. It will be appreciated that the antipsychotic agents when used in combination with the compounds of the invention may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, olanzapine, haloperidol, pimozide and risperidone are commonly used in a non-salt form.

Other classes of antipsychotic agent of use in combination with the compounds of the invention include dopamine receptor antagonists, especially D2, D3 and D4 dopamine receptor antagonists, and muscarinic ml receptor agonists. An example of a D3 dopamine receptor antagonist is the compound PNU-99194A. An example of a D4 dopamine receptor antagonist is PNU-101387. An example of a muscarinic ml receptor agonist is xanomeline.

Another class of antipsychotic agent of use in combination with the compounds of the invention is the 5-HT_{2A} receptor antagonists, examples of which include MDL100907 and fananserin. Also of use in combination with the compound of the invention are the serotonin dopamine antagonists (SDAs) which are believed to combine 5-HT_{2A} and dopamine receptor antagonist activity, examples of which include olanzapine and ziperasidone.

Therefore, in a further aspect of the invention, a pharmaceutical composition is provided comprising a compound of the invention and an antipsychotic agent, together with at least one pharmaceutically acceptable carrier or excipient.

The compounds of the invention and the other pharmacologically active agent may be administered to a patient simultaneously, sequentially or in combination. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

The compounds of the invention may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician.

In the treatment of a condition associated with an excess of tachykinins, an appropriate dosage level will generally be about 0.001 to 50 mg per kg patient body weight per day which may be administered in single or multiple doses. Preferably, the dosage level will be about 0.01 to about 25 mg/kg per day; more preferably about 0.05 to about 10 mg/kg per day. For example, in the treatment of conditions involving the neurotransmission of pain sensations, a suitable dosage level is about 0.001 to 25 mg/kg per day, preferably about 0.005 to 10 mg/kg per day, and especially about 0.01 to 5 mg/kg per day. A compound may be administered on a regimen of 1 to 4 times per day, preferably once or twice per day. In the treatment of emesis, a suitable dosage level is about 0.001 to 10 mg/kg per day, preferably about 0.005 to 5 mg/kg per day, and especially about 0.01 to 1 mg/kg per day. The compound may be administered on a regimen of 1 to 4 times per day, preferably once or twice per day. In the treatment or prevention of a disorder of the central nervous system, a suitable dosage level is about 0.001 to 10 mg/kg per day, preferably about 0.005 to 5 mg/kg per day, and especially about 0.01 to 1 mg/kg per day. The compounds may be administered on a regimen of 1 to 4 times per day, preferably once or twice per day.

It will be appreciated that the amount of the compound of the invention required for use in any treatment will vary not only with the particular compounds or composition selected but also with the route of administration, the nature of the condition being treated, and the age and condition of the patient, and will ultimately be at the discretion of the attendant physician.

The compositions and combination therapies of the invention may be administered in combination with a variety of pharmaceutical excipients, including stabilizing agents, carriers and/or encapsulation formulations as described herein.

Aqueous compositions of the present invention comprise an effective amount of the peptides of the invention, dissolved or dispersed in a pharmaceutically acceptable carrier or aqueous medium.

“Pharmaceutically or pharmacologically acceptable” include molecular entities and compositions that do not produce an adverse, allergic or other untoward reaction when administered to an animal, or a human, as appropriate. “Pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For human administration, preparations should meet sterility, pyrogenicity, general safety and purity standards as required by FDA Office of Biologics standards.

The compositions and combination therapies of the invention will then generally be formulated for parenteral administration, e.g., formulated for injection via the intravenous, intramuscular, subcutaneous, intralesional, or even intraperitoneal routes. The preparation of an aqueous composition that contains a composition of the invention or an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Therapeutic or pharmacological compositions of the present invention will generally comprise an effective amount of the component(s) of the combination therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including cremes, lotions, mouthwashes, inhalants and the like.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly, concentrated solutions for intramuscular injection is also contemplated. In this regard, the use of DMSO as solvent is preferred as this will result in extremely rapid penetration, delivering high concentrations of the active compound(s) or agent(s) to a small area.

The use of sterile formulations, such as saline-based washes, by surgeons, physicians or health care workers to cleanse a particular area in the operating field may also be particularly useful. Therapeutic formulations in accordance with the present invention may also be reconstituted in the form of mouthwashes, or in conjunction with antifungal reagents. Inhalant forms are also envisioned. The therapeutic formulations of the invention may also be prepared in forms suitable for topical administration, such as in cremes and lotions.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals. For example, for parenteral administration, a suitably buffered, and if necessary, isotonic aqueous solution would be prepared and used for intravenous, intramuscular, subcutaneous or even intraperitoneal administration. One dosage could be dissolved in 1 ml of isotonic NaCl solution and either added to 1000 ml of hypodermolysis fluid or injected at the proposed site of infusion, (see for example, Remington's Pharmaceutical Sciences 15th Edition, pages 1035-1038 and 1570-1580).

In certain embodiments, active compounds may be administered orally. This is contemplated for agents which are generally resistant, or have been rendered resistant, to proteolysis by digestive enzymes. Such compounds are contemplated to include chemically designed or modified agents; dextrorotatory peptides; and peptide and liposomal formulations in time release capsules to avoid peptidase and lipase degradation.

Pharmaceutically acceptable salts include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze drying techniques which yield a powder of the active ingredient plus any additional desired ingredient from a previously sterile-filtered solution thereof.

The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure.

In addition to the compounds formulated for parenteral administration, such as intravenous or intramuscular injection, other pharmaceutically acceptable forms include, e.g., tablets or other solids for oral administration; liposomal formulations; time-release capsules; and any other form currently used, including cremes.

Additional formulations suitable for other modes of administration include suppositories. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabensas preservatives, a dye and flavoring, such as cherry or orange flavor.

The pharmaceutical compositions of this invention may be used in the form of a pharmaceutical preparation, for example, in solid, semisolid or liquid form, which contains one or more of the compound of the invention, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an amount sufficient to produce the desired effect upon the process or condition of the disease.

For preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions of the invention may be incorporated for administration orally or by injection include aqueous solution, suitably flavored syrups, aqueous or oil suspensions, and emulsions with acceptable oils such as cottonseed oil, sesame oil, coconut oil or peanut oil, or with a solubilizing or emulsifying agent suitable for intravenous use, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

For treating clinical conditions and diseases noted above, the compound of this invention may be administered orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrastemal injection or infusion techniques.

Methods for preparing the compounds of this invention are illustrated in the following Example(s). The following examples are given for the purpose of illustrating the invention, but not for limiting the scope or spirit of the invention.

### EXAMPLES

The following detailed experimental section sets forth synthetic schemes for producing compounds of the invention, as well as useful intermediate compounds used in the syntheses described herein.

### Example 1

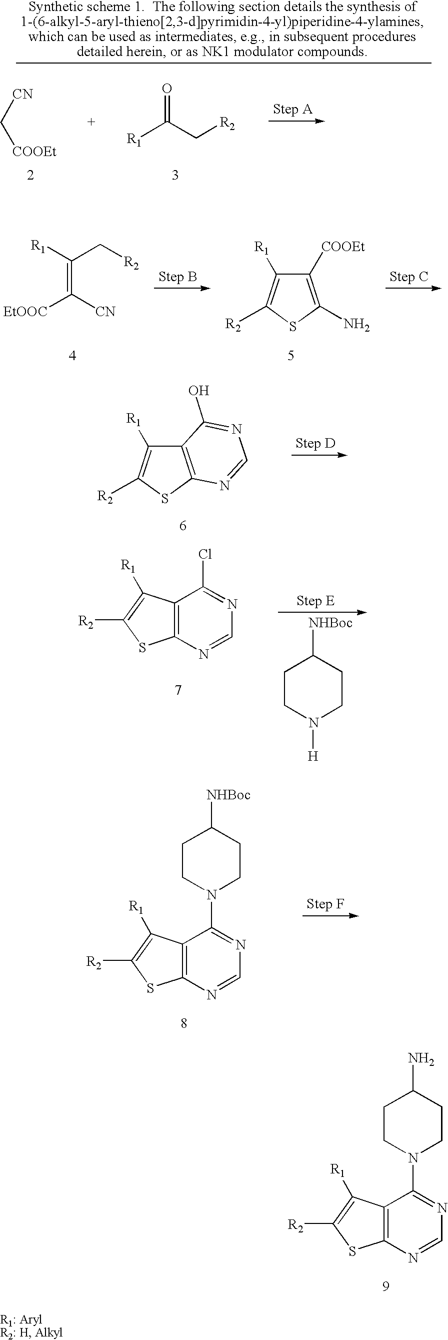

General Procedure—Step A.

To a toluene (200 mL) solution of alkylaryl ketone 2 (166.4 mmol) was added ethyl cyanoacetate 3 (199.74 mmol) and ammonium acetate (332.9 mmol), followed by acetic acid (166.45 mmol). The resulting suspension was refluxed using Dean-Stark for 16 h under nitrogen. After cooling to room temperature, the reaction mixture was concentrated in vacuum. Water was added to the residue, and the product was extracted with ethyl acetate (3×100 ml). The combined organic layer was dried over sodium sulfate and concentrated. The crude product 4 was used as such for the next step (step B).

General Procedure for Step B.

Sulfur (126.53 mmol) was added to a mixture of crude product 4 (57.5 mmol) and morpholine (66.1 mmol) in ethanol (150 ml) under nitrogen atmosphere. The resulting suspension was stirred at reflux for 14 h. After cooling to room temperature, the reaction mixture was concentrated in vacuum. Water was added to the residue, and the product was extracted with ethyl acetate (3×100 ml). The combined organic layer was dried over sodium sulfate and concentrated. A 2-Amino-4-aryl-5-alkyl-thiophene-3-carboxylic acid ethyl ester 5 (shown below) was purified by column chromatography (Hexanes: Ethyl acetate 10:4). Typical yields were 50-70%.

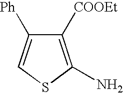

¹H NMR (500 MHz, CDCl₃): δ 7.31 (m, 5H), 6.06 (s, 1 H), 4.05 (q, 2H), 0.93 (t, 3H).

General Procedure for Step C.

The 2-Amino-5-alkyl-4-Aryl-thiophene-3-carboxylic acid ethyl ester 5 (1 mmol) was used as a reactive intermediate in this procedure. The compound was suspended in ammonium formate (1.5 ml), and the reaction mixture was heated at reflux for 12 h. During this time the reaction completion was monitored via TLC. After cooling, the reaction mixture was poured into ice (50 g) to afford a creamy precipitate. The precipitate was collected by filtration, and recrystallized from acetone/water to give product 6, in typical yields of 70-90%. Specific exemplary compounds of product 6 listed below were made by this procedure, e.g.,

5-Phenyl-thieno[2,3-d]pyrimidin-4-ol

6-Methyl-5-phenyl-thieno[2,3-d]pyrimidin-4-ol

5-(4-Bromo-phenyl)-thieno[2,3-d]pyrimidin-4-ol

5-p-Tolyl-thieno[2,3-d]pyrimidin-4-ol

5-(4-Fluoro-phenyl)-thieno[2,3-d]pyrimidin-4-ol

5-Methyl-thieno[2,3-d]pyrimidin-4-ol

Thieno[2,3-d]pyrimidin-4-ol

General Procedure for Step D.

A mixture of a 5-Aryl-6-alkyl-thieno[2,3-d]pyrimidin-4-ol (3.7 mmol) 6, thionyl chloride (5.5 ml) and dry Dimethylformamide (0.5 ml) was heated at reflux for 4 h. After the reaction mixture was cooled, excess thionyl chloride was removed by distillation and 200 g of ice was added. The product was extracted with dichloromethane (3×100 ml). The combined organic layer was dried over sodium sulfate and concentrated. The product was purified by column chromatography (Dichloromethane) to afford a 4-Chloro-5-Aryl-6-alkyl-thieno[2,3-d]-pyrimidine 7 in 80-95% yield. Specific exemplary compounds of product 7 listed below were made by this procedure, e.g.,

4-Chloro-5-phenyl-thieno[2,3-d]pyrimidine

4-Chloro-6-methyl-5-phenyl-thieno[2,3-d]pyrimidine

4-Chloro-5-p-tolyl-thieno[2,3-d]pyrimidine

5-(4-Bromo-phenyl)-4-chloro-thieno[2,3-d]pyrimidine

4-Chloro-5-methyl-thieno[2,3-d]pyrimidine

4-Chloro-thieno[2,3-d]pyrimidine

General Procedure for Step E.

To a isopropanol (4 ml) solution of 5-Aryl-6-alkyl-4-chloro-thieno[2,3-d]pyrimidine (1 mmol) 7 was added Boc-4-amino-piperidine (2 mmol), followed by triethylamine (4 mmol), and the solution was heated at reflux for 2 h. The solvent was evaporated and ether was added to the residue. The ethereal layer containing the crude product was filtered off, and was concentrated to yield a crude product. A 5-Aryl-6-alkyl-thieno[2,3-d]pyrimidin-4-yl]-piperidin-4-yl-carbamic acid tert-butyl ester 8 was obtained in pure form after column chromatography (1% MeOH in Dichloromethane) typical yields were from 75-95%. Specific exemplary compounds of product 8 listed below were made by this procedure, e.g.,

[1-(5-Phenyl-thieno[2,3-d]pyrimidin-4-yl)-piperidin-4-yl]-carbamic acid tert-butyl ester

[1-(6-Methyl-5-phenyl-thieno[2,3-d]pyrimidin-4-yl)-piperidin-4-yl]-carbamic acid tert-butyl ester

{1-[5-(4-Bromo-phenyl)-thieno[2,3-d]pyrimidin-4-yl]-piperidin-4-yl}-carbamic acid tert-butyl ester

[1-(5-p-Tolyl-thieno[2,3-d]pyrimidin-4-yl)-piperidin-4-yl]-carbamic acid tert-butyl ester

[1-(5-Methyl-thieno[2,3-d]pyrimidin-4-yl)-piperidin-4-yl]-carbamic acid tert-butyl ester

(1-Thieno[2,3-d]pyrimidin-4-yl-piperidin-4-yl)-carbamic acid tert-butyl ester

General Procedure for Step F.

HCl gas was passed through dry diethyl ether (50 ml) for a period of 5 min. To this solution was added a solution of a [1-5-aryl-6-alkyl-thieno[2,3-d]pyrimidin-4-yl)-piperidin-4-yl]-carbamic acid tert-butyl ester 8 (0.83 mmol) in dry dichloromethane (2 ml). The suspension was refrigerated overnight. The hydrochloride salt of a 1-[5-aryl-6-alky-lthieno[2,3-d]pyrimidin-4-yl]-piperidin-4-ylamine 9 was obtained by the filtration of the ether layer. The product was dried under vacuum. To the solid, 50 ml ammonium hydroxide was added to release free amine and the aqueous solution was extracted with chloroform (3×50 ml). The combined organic layer was dried and concentrated to afford a 1-[5-aryl-6-alkyl-thieno[2,3-d]pyrimidin-4-yl]-piperidin-4-ylamine 9. Typical yields were 80-95%. Specific exemplary compounds of product 9 listed below were made by this procedure, e.g.,

1-(5-phenyl-theino[2,3-d]pyrimidine-4-yl)-piperidin-4-ylamine

1-(6-Methyl-5-phenyl-thieno[2,3-d]pyrimidin-4-yl)-piperidin-4-ylamine

1-[5-(4-Bromo-phenyl)-thieno[2,3-d]pyrimidin-4-yl]-piperidin-4-ylamine

1-(5-p-Tolyl-thieno[2,3-d]pyrimidin-4-yl)-piperidin-4-ylamine

1-(5-Methyl-thieno[2,3-d]pyrimidin-4-yl)-piperidin-4-ylamine

1-Thieno[2,3-d]pyrimidin-4-yl-piperidin-4-ylamine

### Example 2

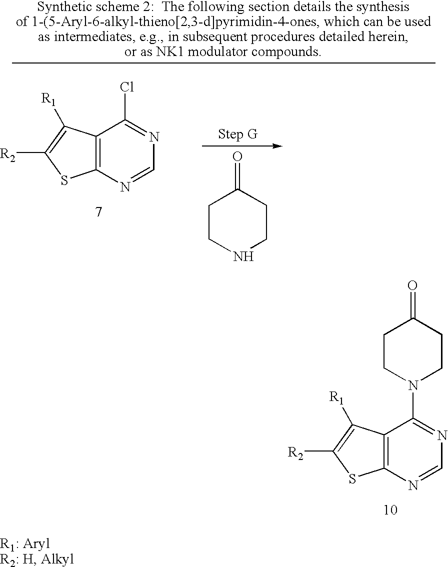

General Procedure for Step G.

To a acetonitrile (6 ml) solution of a 5-aryl-6-alkyl-4-chloro-thieno[2,3-d]pyrimidine 7 (0.25 mmol) was added 4-piperidone hydrochloride (0.5 mmol), followed by triethylamine (1 mmol), and the solution was heated for 2.5 h at reflux. The solvent was evaporated and ether was added to the residue. The ethereal layer which contained crude product was collected after the filtration. Pure product 10 was obtained by column chromatography (2% MeOH in Dichloromethane). Typical yields were 55-70%. Specific exemplary compounds of product 10 listed below were made by this procedure, e.g.,

1-(5-Phenyl-thieno[2,3-d]pyrimidin-4-yl)-piperidin-4-one

1-[5-(4-Bromo-phenyl)-thieno[2,3-d]pyrimidin-4-yl]-piperidin-4-one

1-(5-p-Tolyl-thieno[2,3-d]pyrimidin-4-yl)-piperidin-4-one

1-(6-Methyl-5-phenyl-thieno[2,3-d]pyrimidin-4-yl)-piperidin-4-one

### Example 3

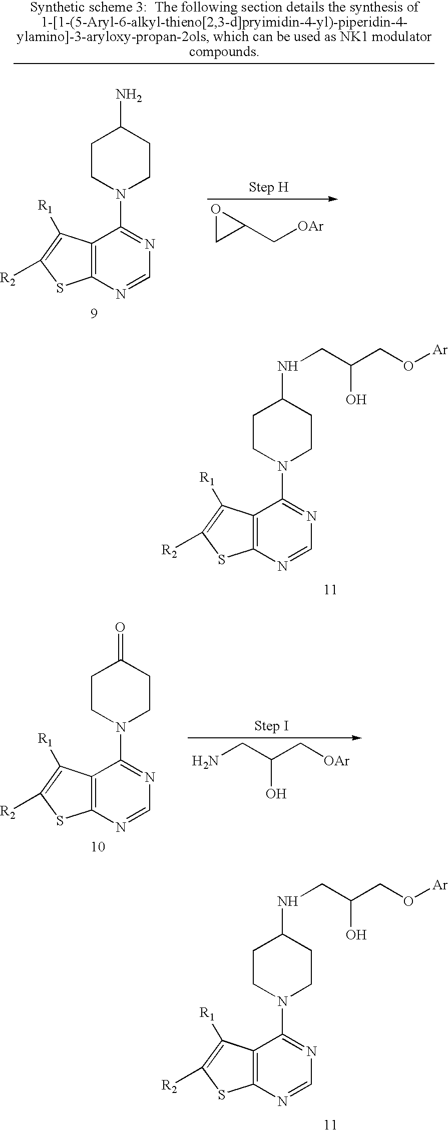

General Procedure for Step H.

A mixture of a 1-(5-aryl-6-alkyl-thieno[2,3-d]pyrimidin-4-yl)-piperidin-4-ylamine (3 mmol) and aryl glycidyl ether 9 (1 mmol) in methanol (5 ml) was refluxed for 3 h. After 3 h, no glycidyl ether was observed by TLC. The reaction mixture was concentrated in vacuum and subjected to column chromatography. On elution with 4% MeOH/DCM, final product 11 was isolated. Typical yields were 50-75%.%. Specific exemplary compounds of product 11 listed below were made by this procedure, e.g.,

1-(4-Methoxy-phenoxy)-3-[1-(5-phenyl-thieno[2,3-d]pyrimidin-4-yl)-piperidin-4-ylamino]-propan-2-ol

2-{1-[5-(4-Bromo-phenyl)-thieno[2,3-d]pyrimidin-4-yl]-piperidin-4-ylamino}-cyclohexanol

2-[1-(5-p-Tolyl-thieno[2,3-d]pyrimidin-4-yl)-piperidin-4-ylamino]-cyclohexanol

2-[1-(6-Methyl-5-phenyl-thieno[2,3-d]pyrimidin-4-yl)-piperidin-4-ylamino]-cyclohexanol

1-(4-Chloro-phenoxy)-3-[1-(5-phenyl-thieno[2,3-d]pyrimidin-4-yl)-piperidin-4-ylamino]-propan-2-ol

1-Phenoxy-3-[1-(5-phenyl-thieno[2,3-d]pyrimidin-4-yl)-piperidin-4-ylamino]-propan-2-ol

1-Benzyloxy-3-[1-(5-phenyl-thieno[2,3-d]pyrimidin-4-yl)-piperidin-4-ylamino]-propan-2-ol

1-(Benzo[1,3]dioxol-5-yloxy)-3-[1-(5-phenyl-thieno[2,3-d]pyrimidin-4-yl)-piperidin-4-ylamino]-propan-2-ol

1-(Benzo[1,3]dioxol-5-ylmethoxy)-3-[1-(5-phenyl-thieno[2,3-d]pyrimidin-4-yl)-piperidin-4-ylamino]-propan-2-ol

1-(3,4-Difluoro-phenoxy)-3-[1-(5-phenyl-thieno[2,3-d]pyrimidin-4-yl)-piperidin-4-ylamino]-propan-2-ol

1-(2-Chloro-4-methoxy-phenoxy)-3-[4-(5-phenyl-thieno[2,3-d]pyrimidin-4-yl)-cyclohexylamino]-propan-2-ol

1-(3,4-Dimethoxy-phenoxy)-3-[4-(5-phenyl-thieno[2,3-d]pyrimidin-4-yl)-cyclohexylamino]-propan-2-ol

1-(3,4-Dichloro-phenoxy)-3-[1-(5-phenyl-thieno[2,3-d]pyrimidin-4-yl)-piperidin-4-ylamino]-propan-2-ol

1-(3-Chloro-4-fluoro-phenoxy)-3-[1-(5-phenyl-thieno[2,3-d]pyrimidin-4-yl)-piperidin-4-ylamino]-propan-2-ol

1-(2,4-Difluoro-phenoxy)-3-[1-(5-phenyl-thieno[2,3-d]pyrimidin-4-yl)-piperidin-4-ylamino]-propan-2-ol

1-(3,5-Difluoro-phenoxy)-3-[1-(5-phenyl-thieno[2,3-d]pyrimidin-4-yl)-piperidin-4-ylamino]-propan-2-ol

1-(3,5-Bis-trifluoromethyl-phenoxy)-3-[1-(5-phenyl-thieno[2,3-d]pyrimidin-4-yl)-piperidin-4-ylamino]-propan-2-ol

1-(Benzo[1,3]dioxol-5-yloxy)-3-[1-(5-methyl-thieno[2,3-d]pyrimidin-4-yl)-piperidin-4-ylamino]-propan-2-ol

1-(Benzo[1,3]dioxol-5-yloxy)-3-(1-thieno[2,3-d]pyrimidin-4-yl-piperidin-4-ylamino)-propan-2-ol

General Procedure for Step I.

A 1-[5-Aryl-6-alkyl-thieno[2,3-d]pyrimidin-4-yl]-piperidin-4-one 10 (0.134 mmol) and 1-amino-2-alkyl(aryl) alcohol (0.134 mmol) were mixed in dry dichloromethane (2 ml) and then treated with sodiumtriacetoxyborohydride (0.2 mmol) and acetic acid (0.134 mmol). The mixture was stirred at room temperature under N₂ atmosphere for 18 h until the reactants were consumed as determined by TLC analysis. The reaction mixture was quenched by addition of 1N NaOH and the product was extracted with dichloromethane. The organic layer was dried over sodium sulfate and concentrated to afford crude product. The crude product was subjected to column chromatography (2% MeOH/DCM) to yield the product 11. Typical yields were from 60-80%. The specific compounds listed below were made by this procedure, e.g.

2-[1-(5-Phenyl-thieno[2,3-d]pyrimidin-4-yl)-piperidin-4-ylamino]-cyclohexanol

2-[1-(6-Methyl-5-phenyl-thieno[2,3-d]pyrimidin-4-yl)-piperidin-4-ylamino]-cyclohexanol

1-[1-(6-Methyl-5-phenyl-thieno[2,3-d]pyrimidin-4-yl)-piperidin-4-ylamino]-indan-2-ol

5-Methoxy-2-{[1-(5-phenyl-thieno[2,3-d]pyrimidin-4-yl)-piperidin-4-ylamino]-methyl}-phenol

Bis-(2-fluoro-benzyl)-[1-(5-phenyl-thieno[2,3-d]pyrimidin-4-yl)-piperidin-4-yl]-amine

1-{-[5-(4-Bromo-phenyl)-thieno[2,3-d]pyrimidin-4-yl]-piperidin-4-ylamino}-indan-2-ol

1-[1-(5-p-Tolyl-thieno[2,3-d]pyrimidin-4-yl)-piperidin-4-ylamino]-indan-2-ol

2-Fluoro-6-{[1-(6-methyl-5-phenyl-thieno[2,3-d]pyrimidin-4-yl)-piperidin-4-ylamino]-methyl}-phenol

2-({1-[5-(4-Bromo-phenyl)-thieno[2,3-d]pyrimidin-4-yl]-piperidin-4-ylamino}-methyl)-6-fluoro-phenol

2-Fluoro-6-{[1-(5-p-tolyl-thieno[2,3-d]pyrimidin-4-yl)-piperidin-4-ylamino]-methyl}-phenol

1-[1-(5-Phenyl-thieno[2,3-d]pyrimidin-4-yl)-piperidin-4-ylamino]-indan-2-ol

1-(4-Fluoro-phenoxy)-3-[1-(5-phenyl-thieno[2,3-d]pyrimidin-4-yl)-piperidin-4-ylamino]-propan-2-ol

1-[1-(5-Phenyl-thieno[2,3-d]pyrimidin-4-yl)-piperidin-4-ylamino]-3-(4-trifluoromethoxy-phenoxy)-propan-2-ol

1-(3,4-Difluoro-phenoxy)-3-{1-[5-(4-fluoro-phenyl)-thieno[2,3-d]pyrimidin-4-yl]-piperidin-4-ylamino}-propan-2-ol

### Example 5

General Procedure for Salt Formation:

Pharmaceutically acceptable salts of compounds of the invention as set forth, e.g., in Scheme 3, maybe formed as follows. A 1-(Aryl)-3-[1-(5-aryl-6-alkyl-thieno[2,3-d]pyrimidin-4-yl)-piperidin-4-ylamino]-propan-2-ol was dissolved in dry dichloromethane (1 mL). This solution was added to 2M HCl solution in ether (10 mL) cooled to −10° C. The suspension was stored at 6° C. overnight. The product was filtered, washed with ether, and dried under vacuum. Typical yields were 90-95%. Specific exemplary compounds listed below were made by this procedure, e.g.,

[2-Hydroxy-3-(4-methoxy-phenoxy)-propyl]-[1-(5-phenyl-thieno[2,3-d]pyrimidin-4-yl)-piperidin-4-yl]-ammonium; chloride

[3-(2-Chloro-4-methoxy-phenoxy)-2-hydroxy-propyl]-[4-(5-phenyl-thieno[2,3-d]pyrimidin-4-yl)-cyclohexyl]-ammonium; chloride

[3-(3,4-Dimethoxy-phenoxy)-2-hydroxy-propyl]-[4-(5-phenyl-thieno[2,3-d]pyrimidin-4-yl)-cyclohexyl]-ammonium; chloride

[3-(3,4-Dichloro-phenoxy)-2-hydroxy-propyl]-[1-(5-phenyl-thieno[2,3-d]pyrimidin-4-yl)-piperidin-4-yl]-ammonium; chloride

[3-(2,4-Difluoro-phenoxy)-2-hydroxy-propyl]-[1-(5-phenyl-thieno[2,3-d]pyrimidin-1-yl)-piperidin-4-yl]-ammonium; chloride

### Example 6

The activity of compounds of the invention were tested in vitro for binding to NK₁ and NK2 receptors. A number of them show very good activity and selectivity, e.g., as NK₁ modulators. These results appear in Table 1, below.

TABLE 1

| Compound | Kᵢ (NK₁) | Kᵢ (NK₂) |
|---|---|---|
| 1-(Benzo[1,3]dioxol-5-yloxy)-3-[1-(5-phenyl-thieno[2,3- | 7.6 nM | >10000 |
| d]pyrimidin-4-yl)-piperidin-4-ylamino]-propan-2-ol | | |
| 1-(4-Methoxy-phenoxy)-3-[1-(5-phenyl-thieno[2,3-d]pyrimidin-4- | 53 nM | 1.7 μM |
| yl)-piperidin-4-ylamino]-propan-2-ol | | |
| [3-(4-Chloro-phenoxy)-2-hydroxy-propyl]-[1-(5-phenyl-thieno[2,3- | 910 nM | >>10000 |
| d]pyrimidin-4-yl)-piperidin-4-yl]-ammonium; chloride | | |
| 1-Phenoxy-3-[1-(5-phenyl-thieno[2,3-d]pyrimidin-4-yl)-piperidin-4- | 841 nM | >10000 |
| ylamino]-propan-2-ol | | |
| (3-Benzyloxy-2-hydroxy-propyl)-[1-(5-phenyl-thieno[2,3- | 141 nM | 2.4 μM |
| d]pyrimidin-4-yl)-piperidin-4-yl]-ammonium; chloride | | |
| 1-(4-Chloro-3-methoxy-phenoxy)-3-[1-(5-phenyl-thieno[2,3- | 126 nM | 47% @ |
| d]pyrimidin-4-yl)-piperidin-4-ylamino]-propan-2-ol | | 1000 |
| 1-(3,4-Dimethoxy-phenoxy)-3-[1-(5-phenyl-thieno[2,3-d]pyrimidin- | 126 nM | 6250 |
| 4-yl)-piperidin-4-ylamino]-propan-2-ol | | |
| 1-(3,4-Dichloro-phenoxy)-3-[1-(5-phenyl-thieno[2,3-d]pyrimidin-4- | 268 nM | 3390 |
| yl)-piperidin-4-ylamino]-propan-2-ol | | |
| [3-(3,4-Dichloro-phenoxy)-2-hydroxy-propyl]-[1-(5-phenyl- | 20 nM | 28% @ |
| thieno[2,3-d]pyrimidin-4-yl)-piperidin-4-yl]-ammonium; chloride | | 1000 |
| [2-Hydroxy-3-(4-trifluoromethyl-phenoxy)-propyl]-[1-(5-phenyl- | 53 nM | 14% @ |
| thieno[2,3-d]pyrimidin-4-yl)-piperidin-4-yl]-ammonium; chloride | | 1000 |
| 1-(4-Fluoro-phenoxy)-3-[1-(5-phenyl-thieno[2,3-d]pyrimidin-4-yl)- | 24 nM | 22% @ |
| piperidin-4-ylamino]-propan-2-ol | | 1000 |
| 1-[1-(5-Phenyl-thieno[2,3-d]pyrimidin-4-yl)-piperidin-4-ylamino]-3- | 15 nM | 49% @ |
| (4-trifluoromethoxy-phenoxy)-propan-2-ol | | 1000 |
| 1-(2,4-Difluoro-phenoxy)-3-[1-(5-phenyl-thieno[2,3-d]pyrimidin-4- | 286 nM | — |
| yl)-piperidin-4-ylamino]-propan-2-ol | | |
| 1-(3,5-Bis-trifluoromethyl-phenoxy)-3-[1-(5-phenyl-thieno[2,3- | 1310 nM | >1000 |
| d]pyrimidin-4-yl)-piperidin-4-ylamino]-propan-2-ol | | |
| 1-(3-Chloro-4-fluoro-phenoxy)-3-[1-(5-phenyl-thieno[2,3- | 137 nM | — |
| d]pyrimidin-4-yl)-piperidin-4-ylamino]-propan-2-ol | | |
| 1-(3,4-Difluoro-phenoxy)-3-[1-(5-phenyl-thieno[2,3-d]pyrimidin-4- | 75 nM | 28% @ |
| yl)-piperidin-4-ylamino]-propan-2-ol | | 1000 |
| 1-(Benzo[1,3]dioxol-5-yloxy)-3-[1-(5-methyl-thieno[2,3- | 868 nM | — |
| d]pyrimidin-4-yl)-piperidin-4-ylamino]-propan-2-ol | | |
| 1-(2-Chloro-4-methoxy-phenoxy)-3-[1-(5-phenyl-thieno[2,3- | 109 nM | 1570 |
| d]pyrimidin-4-yl)-piperidin-4-ylamino]-propan-2-ol | | |
| 1-(3,5-Difluoro-phenoxy)-3-[1-(5-phenyl-thieno[2,3-d]pyrimidin-4- | 272 nM | 21% @ |
| yl)-piperidin-4-ylamino]-propan-2-ol | | 1000 |
| 4-{4-[2-(4-Fluoro-phenoxymethyl)-morpholin-4-yl]-piperidin-1-yl}- | 472 nM | — |
| 5-phenyl-thieno[2,3-d]pyrimidine | | |
| 4-{4-[2-(Benzo[1,3]dioxol-5-yloxymethyl)-morpholin-4-yl]- | 1.86 μM | — |
| piperidin-1-yl}-5-phenyl-thieno[2,3-d]pyrimidine | | |
| 1-(Benzo[1,3]dioxol-5-yloxy)-3-(1-thieno[2,3-d]pyrimidin-4-yl- | 1.83 μM | — |
| piperidin-4-ylamino)-propan-2-ol | | |
| 6-(Benzo[1,3]dioxol-5-yloxymethyl)-4-[1-(5-phenyl-thieno[2,3- | 1.32 μM | — |
| d]pyrimidin-4-yl)-piperidin-4-yl]-morpholin-3-one | | |

Equivalents

Those skilled in the art will recognize, or be able to ascertain using no more than routine experimentation, numerous equivalents to the specific procedures described herein. Such equivalents are considered to be within the scope of the invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby incorporated by reference. The appropriate components, processes, and methods of those patents, applications and other documents may be selected for the invention and embodiments thereof.

What is claimed is:

1. A compound having the formula 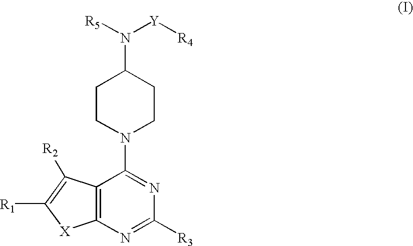
wherein
X is S, O, C, NH, NR, or NCOR;
R₁ and R₂ each independently are H; (C₁-C₇)alkyl; (C₁-C₇)cycloalkyl; (CH₂)ₙ—(C₁-C₇)cycloalkyl, aryl, substituted aryl, heteroaryl, or substituted heteroaryl; or R₁ and R₂, when joined by a single or multiple bonds, can form an aliphatic or an aromatic ring;
R₃ is H, (C₁-C₄)alkyl, (C₁-C₆)cycloalkyl, aryl, substituted aryl, heteroaryl or substituted heteroaryl;
R₄ is H, (C₁-C₅)alkyl, (C₁-C₆)cycloalkyl, aryl, substituted aryl, heteroaryl, or substituted heteroaryl; (CH₂)ₙ₋aryl or (CH₂)ₙ-heteroaryl, where n is 1, 2 or 3;
Y is CH₂, hydroxycyclohexyl, 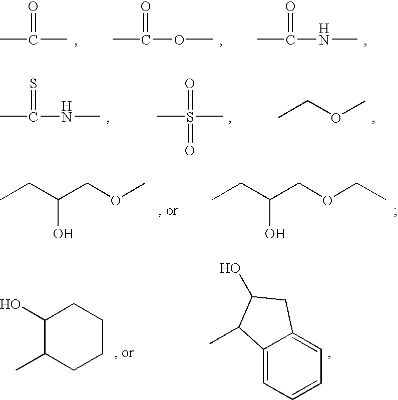
with the proviso that when R₅ forms a heterocyclic ring with the nitrogen to which it is attached, Y is attached to the heterocylic ring;
R₅ is H; (C₁-C₅)alkyl; (C₁-C₆)cycloalkyl, aryl, substituted aryl, heteroaryl or substituted heteroaryl; (CH₂)ₙ₋aryl or (CH₂)ₙ-heteroaryl, where n is 1, 2 or 3; 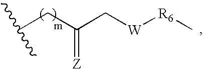
where m is 1, 2, 3, 4 or 5; or R₅, taken with the nitrogen to which it is attached, forms a five or six membered heterocyclic ring to which Y is attached, of the structure 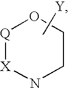
where X is a methylene (—CH₂—) or carbonyl group 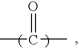
and Q is a methylene group or not present;
Z is H, H; O, H and OH, O-alkyl where alkyl is (C₁-C₆)alkyl, (C₁-C₆)cycloalkyl, O-alkylaryl, O-benzyl, O—CO-aryl, N—Me, N-acyl, N-aryl, N-aroyl, N—SO₂-alkyl, or N—SO₂-aryl;
W is C, O, NH, NR; and
R₆ is H; (C₁-C₅)alkyl; (C₁-C₆)cycloalkyl, aryl, substituted aryl, heteroaryl, or substituted heteroaryl; (CH₂)ₙ₋aryl or (CH₂)ₙ-heteroaryl; where n is 1, 2 or 3; and pharmaceutically acceptable salts and/or esters thereof.

2. The compound of claim 1, wherein said aryl group is selected from the group consisting of phenyl, naphthyl, and biphenyl.

3. The compound of claim 1, wherein said heteroaryl group is selected from the group consisting of thiazole, oxazole, benzothiazole, benzoxazole, pyrazole, indole, and indazole.

4. The compound of claim 1, wherein said substituted aryl group is selected from the group consisting of mono-, di-, or tri-substituted phenyl, naphthyl, or biphenyl with methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy.

5. The compound of claim 1, wherein said substituted aryl group is selected from the group consisting of mono-, di-, or tri-substituted thiazole, oxazole, benzothiazole, benzoxazole, pyrazole, indole, and indazole.

6. The compound of claim 5, wherein said substituent is selected from the group consisting of methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy.

7. The compound of claim 1, wherein X is sulfur.

8. A pharmaceutical composition comprising the compound of claim 1 in an amount effective to treat a respiratory disorder.

9. The pharmaceutical composition of claim 8, wherein said respiratory disorder is asthma.

10. A pharmaceutical composition comprising the compound of claim 1 in an amount effective to treat an inflammatory disorder.

11. A pharmaceutical composition comprising the compound of claim 1 in an amount effective to treat inflammation.

12. A pharmaceutical composition comprising the compound of claim 1 in an amount effective to treat inflammation in a mammal suffering therefrom.

13. A pharmaceutical composition comprising the compound of claim 1 in an amount effective to treat a gastrointestinal disorder.

14. The pharmaceutical composition of claim 13, wherein said gastrointestinal disorder is selected from the group consisting of Crohn's disease; colitis; and irritable bowel syndrome.

15. A pharmaceutical composition comprising the compound of claim 1 in an amount effective to treat an opthalmic disease.

16. The pharmaceutical composition of claim 15, wherein said opthalmic disease is selected from the group consisting of glaucoma; dry eye; conjunctivitis; and ocular hypotension.

17. A pharmaceutical composition comprising the compound of claim 1 in an amount effective to treat a condition selected from the group consisting of neural injury; schizophrenia; stroke; psoriasis; an allergic condition; rhinitis and eczema; a CNS disorder; migraines; inflammatory pain; anxiety or depression; emesis; cancer chemotherapy-induced emesis; rheumatoid arthritis; tumor cell growth; and atherosclerosis.

18. A method of treating a respiratory disorder, comprising administering to a patient in need thereof a pharmaceutical composition comprising the compound of claim 1 in an amount effective to treat said respiratory disorder.

19. A method of treating an inflammatory disorder, comprising administering to a patient in need thereof a pharmaceutical composition comprising the compound of claim 1 in an amount effective to treat said inflammatory disorder.

20. A method of treating inflammation, comprising administering to a patient in need thereof a pharmaceutical composition comprising the compound of claim 1 in an amount effective to treat said inflammation.

21. A method of treating a gastrointestinal disorder, comprising administering to a patient in need thereof a pharmaceutical composition comprising the compound of claim 1 in an amount effective to treat said gastrointestinal disorder.

22. A method of treating an opthalmic disease, comprising administering to a patient in need thereof a pharmaceutical composition comprising the compound of claim 1 in an amount effective to treat said opthalmic disease.

23. A method of treating an allergic condition, comprising administering to a patient in need thereof a pharmaceutical composition comprising the compound of claim 1 in an amount effective to treat said allergic condition.

24. A method of treating a CNS disorder, comprising administering to a patient in need thereof a pharmaceutical composition comprising the compound of claim 1 in an amount effective to treat said CNS disorder.

25. A compound having the formula 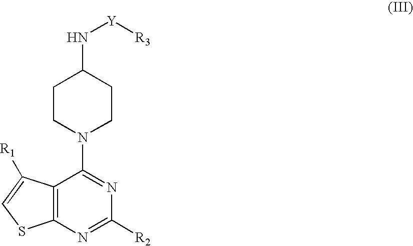
wherein
R₁ is selected from the group consisting of substituted or unsubstituted aryl and substituted or unsubstituted heteroaryl;
R₂ is H, (C₁-C₅)alkyl, (C₁-C₆)cycloalkyl, aryl, substituted aryl, heteroaryl, or substituted heteroaryl; (CH₂)ₙ₋aryl or (CH₂)ₙ-heteroaryl, where n is 1, 2 or 3; and pharmaceutically acceptable salts and/or esters thereof.
R₃ is selected from the group consisting of substituted or unsubstituted aryl and substituted or unsubstituted heteroaryl; and
Y is 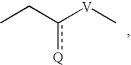
wherein Q or V is O, OH, S, or SH; and pharmaceutically acceptable salts and/or esters thereof.

26. The compound of claim 25, wherein said aryl group is selected from the group consisting of phenyl, naphthyl, and biphenyl.

27. The compound of claim 25, wherein said heteroaryl group is selected from the group consisting of thiazole, oxazole, benzothiazole, benzoxazole, pyrazole, indole, and indazole.

28. The compound of claim 25, wherein said substituted aryl group is selected from the group consisting of mono-, di-, or tri-substituted phenyl, naphthyl, or biphenyl with methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy.

29. The compound of claim 25, wherein said substituted aryl group is selected from the group consisting of mono-, di-, or tri-substituted thiazole, oxazole, benzothiazole, benzoxazole, pyrazole, indole, and indazole.

30. The compound of claim 30, wherein said substituent is selected from the group consisting of methyl, ethyl, propyl, allyl, n-butyl, n-pentyl, n-hexyl, methoxy, ethoxy, propoxy, butoxy, pentyloxy, hexyloxy, cyclopropoxy, cyclopentyloxy, phenoxy, benzyloxy, phenylethoxy, fluoro, chloro, bromo, iodo, amino, dimethylamino, nitro, cyano, trifluoromethyl, trifluoromethoxy, tetrazolo, sulphonyl, thiomethyl, thioethyl, phenylthio, 2,3-methylenedioxy, and 3,4-methylenedioxy.

31. A compound having the formula 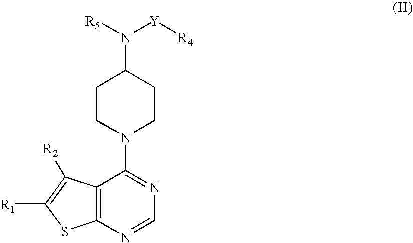
wherein
R₁ is H or CH₃;
R₂ is CH₃ or substituted or unsubstituted aryl;
R₃ is H; (C₁-C₅)alkyl; or substituted or unsubstituted aryl;
Y is CH₂, hydroxycyclohexyl, 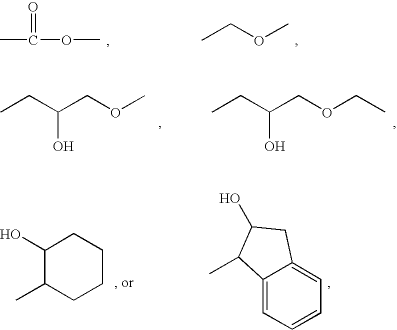
with the proviso that when R₅ forms a heterocyclic ring with the nitrogen to which it is attached, Y is attached to the heterocylic ring;
R₄ is substituted or unsubstituted aryl, e.g., mono-, di- or trisubstituted with halo, trihalomethyl, hydroxyl, alkoxy (e.g., methoxy), or with a dioxole ring; and pharmaceutically acceptable salts and/or esters thereof; and
R₅ is H; (C₁-C₅)alkyl; (C₁-C₆)cycloalkyl, aryl, substituted aryl, heteroaryl or substituted heteroaryl; (CH₂)ₙ₋aryl or (CH₂)ₙ-heteroaryl, where n is 1, 2 or 3; or R₅, taken with the nitrogen to which it is attached, forms a five or six membered heterocyclic ring to which Y is attached, of the structure 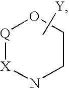
where X is a methylene (—CH₂—) or carbonyl group 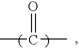
and Q is a methylene group or not present; and pharmaceutically acceptable salts and/or esters thereof.

32. The compound of claim 31, wherein R₄ is mono-, di- or trisubstituted with halo, trihalomethyl, hydroxyl, alkoxy, or with a dioxole ring.

33. A compound selected from the group consisting of 1-(4-Methoxy-phenoxy)-3-[1-(5-phenyl-thieno[2,3-d]pyrimidin-4-yl)-piperidin-4-ylamino]-propan-2-ol; 2-{1-[5-(4-Bromo-phenyl)-thieno[2,3-d]pyrimidin-4-yl]-piperidin-4-ylamino}-cyclohexanol; 2-[1-(5-p-Tolyl-thieno[2,3-d]pyrimidin-4-yl)-piperidin-4-ylamino]-cyclohexanol; 2-[1-(6-Methyl-5-phenyl-thieno[2,3-d]pyrimidin-4-yl)-piperidin-4-ylamino]-cyclohexanol; 1-(4-Chloro-phenoxy)-3-[1-(5-phenyl-thieno[2,3-d]pyrimidin-4-yl)-piperidin-4-ylamino]-propan-2-ol; and pharmaceutically acceptable salts and/or esters thereof.

34. A compound selected from the group consisting of 1-Phenoxy-3-[1-(5-phenyl-thieno[2,3-d]pyrimidin-4-yl)-piperidin-4-ylamino]-propan-2-ol; 1-Benzyloxy-3-[1-(5-phenyl-thieno[2,3-d]pyrimidin-4-yl)-piperidin-4-ylamino]-propan-2-ol; 1-(Benzo[1,3]dioxol-5-yloxy)-3-[1-(5-phenyl-thieno[2,3-d]pyrimidin-4-yl)-piperidin-4-ylamino]-propan-2-ol; 1-(Benzo[1,3]dioxol-5-ylmethoxy)-3-[1-(5-phenyl-thieno[2,3-d]pyrimidin-4-yl)-piperidin-4-ylamino]-propan-2-ol; 1-(3,4-Difluoro-phenoxy)-3-[1-(5-phenyl-thieno[2,3-d]pyrimidin-4-yl)-piperidin-4-ylamino]-propan-2-ol; and pharmaceutically acceptable salts and/or esters thereof.

35. A compound selected from the group consisting of 1-(2-Chloro-4-methoxy-phenoxy)-3-[4-(5-phenyl-thieno[2,3-d]pyrimidin-4-yl)-cyclohexylamino]-propan-2-ol; 1-(3,4-Dimethoxy-phenoxy)-3-[4-(5-phenyl-thieno[2,3-d]pyrimidin-4-yl)-cyclohexylamino]-propan-2-ol; 1-(3,4-Dichloro-phenoxy)-3-[1-(5-phenyl-thieno[2,3-d]pyrimidin-4-yl)-piperidin-4-ylamino]-propan-2-ol; 1-(3-Chloro-4-fluoro-phenoxy)-3-[1-(5-phenyl-thieno[2,3-d]pyrimidin-4-yl)-piperidin-4-ylamino]-propan-2-ol; 1-(2,4-Difluoro-phenoxy)-3-[1-(5-phenyl-thieno[2,3-d]pyrimidin-4-yl)-piperidin-4-ylamino]-propan-2-ol; and pharmaceutically acceptable salts and/or esters thereof.

36. A compound selected from the group consisting of 1-(3,5-Difluoro-phenoxy)-3-[1-(5-phenyl-thieno[2,3-d]pyrimidin-4-yl)-piperidin-4-ylamino]-propan-2-ol; 1-(3,5-Bis-trifluoromethyl-phenoxy)-3-[1-(5-phenyl-thieno[2,3-d]pyrimidin-4-yl)-piperidin-4-ylamino]-propan-2-ol; 1-(Benzo[1,3]dioxol-5-yloxy)-3-[1-(5-methyl-thieno[2,3-d]pyrimidin-4-yl)-piperidin-4-ylamino]-propan-2-ol; 1-(Benzo[1,3]dioxol-5-yloxy)-3-(1-thieno[2,3-d]pyrimidin-4-yl-piperidin-4-ylamino)-propan-2-ol; 2-[1-(5-Phenyl-thieno[2,3-d]pyrimidin-4-yl)-piperidin-4-ylamino]-cyclohexanol; and pharmaceutically acceptable salts and/or esters thereof.

37. A compound selected from the group consisting of 2-[1-(6-Methyl-5-phenyl-thieno[2,3-d]pyrimidin-4-yl)-piperidin-4-ylamino]-cyclohexanol; 1-[1-(6-Methyl-5-phenyl-thieno[2,3-d]pyrimidin-4-yl)-piperidin-4-ylamino]-indan-2-ol; 5-Methoxy-2-{[1-(5-phenyl-thieno[2,3-d]pyrimidin-4-yl)-piperidin-4-ylamino]-methyl}-phenol; Bis-(2-fluoro-benzyl)-[1-(5-phenyl-thieno[2,3-d]pyrimidin-4-yl)-piperidin-4-yl]-amine; 1-{1-[5-(4-Bromo-phenyl)-thieno[2,3-d]pyrimidin-4-yl]-piperidin-4-ylamino}-indan-2-ol; and pharmaceutically acceptable salts and/or esters thereof.

38. A compound selected from the group consisting of 1-[1-(5-p-Tolyl-thieno[2,3-d]pyrimidin-4-yl)-piperidin-4-ylamino]-indan-2-ol; 2-Fluoro-6-{[1-(6-methyl-5-phenyl-thieno[2,3-d]pyrimidin-4-yl)-piperidin-4-ylamino]-methyl}-phenol; 2-({1-[5-(4-Bromo-phenyl)-thieno[2,3-d]pyrimidin-4-yl]-piperidin-4-ylamino}-methyl)-6-fluoro-phenol; 2-Fluoro-6-{[1-(5-p-tolyl-thieno[2,3-d]pyrimidin-4-yl)-piperidin-4-ylamino]-methyl}-phenol; 1-[1-(5-Phenyl-thieno[2,3-d]pyrimidin-4-yl)-piperidin-4-ylamino]-indan-2-ol; and pharmaceutically acceptable salts and/or esters thereof.

39. A compound selected from the group consisting of 1-(4-Fluoro-phenoxy)-3-[1-(5-phenyl-thieno[2,3-d]pyrimidin-4-yl)-piperidin-4-ylamino]-propan-2-ol; 1-[1-(5-Phenyl-thieno[2,3-d]pyrimidin-4-yl)-piperidin-4-ylamino]-3-(4-trifluoromethoxy-phenoxy)-propan-2-ol; 1-(3,4-Difluoro-phenoxy)-3-{1-[5-(4-fluoro-phenyl)-thieno[2,3-d]pyrimidin-4-yl]-piperidin-4-ylamino}-propan-2-ol; [2-Hydroxy-3-(4-methoxy-phenoxy)-propyl]-[1-(5-phenyl-thieno[2,3-d]pyrimidin-4-yl)-piperidin-4-yl]-ammonium; chloride; and pharmaceutically acceptable salts and/or esters thereof.

40. A compound selected from the group consisting of [3-(2-Chloro-4-methoxy-phenoxy)-2-hydroxy-propyl]-[4-(5-phenyl-thieno[2,3-d]pyrimidin-4-yl)-cyclohexyl]-ammonium; chloride; [3-(3,4-Dimethoxy-phenoxy)-2-hydroxy-propyl]-[4-(5-phenyl-thieno[2,3-d]pyrimidin-4-yl)-cyclohexyl]-ammonium; chloride; [3-(3,4-Dichloro-phenoxy)-2-hydroxy-propyl]-[1-(5-phenyl-thieno[2,3-d]pyrimidin-4-yl)-piperidin-4-yl]-ammonium; chloride; [3-(2,4-Difluoro-phenoxy)-2-hydroxy-propyl]-[1-(5-phenyl-thieno[2,3-d]pyrimidin-4-yl)-piperidin-4-yl]-ammonium; chloride; and pharmaceutically acceptable salts and/or esters thereof.
